SEQUENCE LISTING (18 sequences)

SEQ ID NO: 1 - DNA, 60 bp, Sus scrofa domesticus
gatctctggttttggtttcaggaactttgctgccacaattcctcggcccttctcagttcc

SEQ ID NO: 2 - protein, 13 aa, Sus scrofa domesticus
NFAATIPRPFSVP

SEQ ID NO: 3 - DNA, 60 bp, Sus scrofa domesticus
ctagagaccaaaaccaaagtccttgaaacgacggtgttaaggagccgggaagagtcaagc

SEQ ID NO: 4 - DNA, 60 bp, Sus scrofa domesticus
ggtcttaggaactttgctgccacaatacctcggcccttctcagttcgctacgacccatac

SEQ ID NO: 5 - protein, 17 aa, Sus scrofa domesticus
NFAATIPRPFSVRYDPY

SEQ ID NO: 6 - DNA, 60 bp, Sus scrofa domesticus
ggtttcaggaactttgctgccacaattcctcggcccttctcagttcgttatgacccatac

SEQ ID NO: 7 - protein, 17 aa, Sus scrofa domesticus
NFAATIPRPFSVRYDPY

SEQ ID NO: 8 - DNA, 60 bp, Sus scrofa domesticus
ggtcttaggaactttgctgccacaataccttggcccttctcagttcgctacgacccatac

SEQ ID NO: 9 - protein, 17 aa, Sus scrofa domesticus
NFAATIPWPFSVRYDPY

SEQ ID NO: 10 - DNA, 60 bp, Sus scrofa domesticus; identical to SEQ ID NO: 8

SEQ ID NO: 11 - protein, 17 aa, Sus scrofa domesticus
NFAATIPWPFSVRYDPY

SEQ ID NO: 12 - DNA, 36 bp, Homo sapiens
aactttgctgccacaataccttggcccttctcagtt

SEQ ID NO: 13 - protein, 12 aa, Homo sapiens
NFAATIPWPFSV

SEQ ID NO: 14 - DNA, 36 bp, Artificial Sequence
  Synthetic Primer
aactttgctgccacaataccgagacccttctcagtt

SEQ ID NO: 15 - protein, 12 aa, Artificial Sequence
  Synthetic Primer
NFAATIPRPFSV

SEQ ID NO: 16 - DNA, 90 bp, Artificial Sequence
  “donor oligonucleotide”-synthetic DNA
tctcagatctctggttttggtcttaggaactttgctgccacaataccttggcccttctcagttcgctacgacccatacacccaaaggatt

SEQ ID NO: 17 - DNA, 20 bp, Artificial Sequence
  TALEN primers (synthetic)
cttcacctctcagcctggtc

SEQ ID NO: 18 - DNA, 20 bp, Artificial Sequence
  TALEN primers (synthetic)
tgccacgtttcgttctctca

(19) United States
(12) Patent Application Publication
(10) Pub. No.: US 2020/0407755 A1
(43) Pub. Date: Dec. 31, 2020

(54) METHOD AND VECTORS FOR INTRODUCING A GENETIC MUTATION INTO A NON-HUMAN ANIMAL USING A HUMANIZED GENETIC CONSTRUCT

(71) Applicant: Children's Health Care d/b/a Children's Minnesota, Minneapolis, MN (US)

(72) Inventors: Joseph Lillegard, Minneapolis, MN (US); Daniel F. Carlson, St. Paul, MN (US); Cary O. Harding, Portland, OR (US); Dennis Webster, St. Paul, MN (US)

(21) Appl. No.: 16/862,848

(22) Filed: Apr. 30, 2020

Related U.S. Application Data
(60) Provisional application No. 62/840,646, filed on Apr. 30, 2019.

(51) Int. Cl. C12N 15/90; C12N 9/22; A01K 67/027

(52) U.S. Cl. CPC C12N 15/907; C12N 9/22; A01K 67/0275; A01K 2267/035; C12N 2310/20; A01K 2217/07; A01K 2227/108; C12N 2800/80

(57) ABSTRACT

Methods and compositions for introducing genetic mutations into non-human animal cells are provided. These cells can be used to produce animal models of human disease. In some embodiments, the genetic mutations are flanked by DNA sequences that are “humanized” to match homologous DNA sequences. In some embodiments, the animal model is a large mammalian model for an inherited metabolic disorder. In some embodiments, the animal model is a pig model for phenylketonuria (PKU) created by introducing a missense mutation into exon 8 of the Pah gene.

### SEQUENCE LISTING SUBMISSION

The present application includes a Sequence Listing in electronic format as a txt file titled “Sequence-Listing-00562-0006USU1,” which was created on Aug. 17, 2020 and has a size of 3.86 kilobytes (KB). The contents of txt file “Sequence-Listing-00562-0006USU1” are incorporated by reference herein.

### BACKGROUND

Phenylketonuria (PKU) is the most common of the inborn errors of metabolism of the liver, affecting approximately 1 in every 16,000 live births. Typically it is the result of deficiencies in phenylalanine hydroxylase (PAH) activity, which catalyzes the conversion of phenylalanine to tyrosine, arising from genetic mutation. Untreated, PKU causes behavioral problems, neurocognitive impairment, and can eventually become irreversible and progress to seizures.

Therapy involves restricting dietary phenylalanine and/or administering cofactors for PAH (such as tetra hydrobiopterin in the case of partial-activity alleles). Enzyme replacement therapy or orthoptopic liver transplantation are expensive options with lifelong implications that are often not necessary for typical patients. However, there is currently no cure for PKU.

Large animal models can be used to study inborn metabolic disorders such as PKU by introducing genetic mutations into the DNA of the animals to mimic the disorder. Gene editing techniques can be tested on such animal models by modifying the gene editing system to target the sequences found in that particular animal. However, these modifications make it more difficult to accurately test techniques that will be used to treat humans.

### SUMMARY

In general terms, this disclosure is directed to methods and compositions for introducing genetic mutations into non-human animal cells. These cells can be used to produce animal models of human disease. In some embodiments, the genetic mutations are flanked by DNA sequences that are “humanized” to match homologous human DNA sequences. In some embodiments, the animal model is a large mammalian model for an inherited metabolic disorder.

In one aspect, a method of introducing a mutation into a target gene in a non-human animal cell is described. A targeted nuclease system is designed to introduce double-stranded breaks in the DNA flanking the target gene. A homology-directed repair (HDR) template oligonucleotide is designed to include a DNA sequence encoding a mutation in the target gene flanked by DNA sequences homologous to human DNA sequences flanking the mutation. The targeted nuclease system and HDR template are transduced into the non-human animal cell.

In another aspect, a transgenic non-human animal cell has a genome with a DNA sequence encoding a mutation in a gene of interest; and flanking sequences having one or more single nucleotide polymorphisms (SNPs) that cause at least 20 nucleotides upstream and downstream of the mutation to be homologous with human DNA.

In another aspect, a recombinant vector includes a polynucleotide. The polynucleotide encodes a targeted nuclease system designed to introduce double-stranded breaks in the DNA flanking a gene of interest in a non-human animal; and a homology-directed repair (HDR) template having a DNA sequence including a mutation in the gene of interest that modifies its function and flanking sequences mutated to be homologous to human DNA flanking the location of the mutation.

In yet another aspect, a non-human mammal model of an inborn error of metabolism has a DNA sequence including a mutation in a gene that causes the inborn error of metabolism, and two or more single nucleotide polymorphisms (SNPs) in the DNA flanking the mutation that causes the DNA to match a homologous human DNA sequence.

In another aspect, a method of producing a porcine model of phenylketonuria comprises: transferring a nucleus from a donor cell to a denucleated pig oocyte to create a transgenic embryo, the nucleus comprising DNA encoding a Pah gene having a mutation causing loss of function and flanking regions surrounding the mutation that are homologous to human DNA; and implanting the transgenic embryo into a sow.

### BRIEF DESCRIPTION OF THE DRAWINGS

FIG. 1 illustrates a schematic diagram outlining a method of introducing a gene mutation flanked by humanized DNA sequences into the genome of a non-human animal.

FIG. 2 illustrates a schematic diagram outlining a method of transferring the modified DNA sequence of FIG. 1 into an embryo to produce a model animal.

FIG. 3 is a schematic diagram illustrating the design of TALENs to target Exon 8 of Sus scrofa (ss) PAH.

FIG. 4 shows a sequence alignment of human (hs, top) and wild type pig (ss, second) PAH sequences indicating high similarity. A homology template (HT, third) was designed to produce the R408W mutation in addition to introducing the 5 SNPs (black boxes) needed to “humanize” the sequence around the mutation to allow for targeting with human-translatable gene editing reagents. PAH KO shows a representative piglet chromatogram that confirms that the piglets were positive for R408W and the 5 humanizing SNPs.

FIG. 5 is a graph showing birth weights of piglets. Birth weights of PAH KO piglets are lower than historical values for wild type piglets of their respective genetic background strains. Gestation on NTBC showed higher birth weights, similar to reference values, in the single Large White/Landrace litter produced.

FIG. 6 is a bar graph indicating the level of circulating phenylalanine (Phe) and tyrosine (Tyr) in the pigs. Three large white/landrace founder PAH KO piglets maintained on nitisinone (NTBC) were analyzed at birth, with the sow being analyzed at the time of C-section for comparison. Wild type tyrosine level is represented for comparison due to the elevated levels present in the sow, as maintained on NTBC.

FIG. 7 shows two graphs illustrating serum levels of tyrosine in two live-born piglets that were maintained for 6 days (Charm) or continuously (Lucky) to characterize the R408W metabolic phenotype. Tyrosine levels (blue) were elevated at birth due to NTBC administration during gestation, and showed muted responsiveness to phenylalanine levels during development.

FIG. 8 shows two graphs illustrating serum levels of phenylalanine in the two live-born piglets, Charm and Lucky. Serum phenylalanine levels (red) show fluctuation responsive to dietary phenylalanine available (green), indicative of disrupted phenylalanine metabolism and modeling the human disease.

FIG. 9 is a bar graph representing ALP and AST levels in IU/L of pig Lucky. Liver enzyme analysis of Lucky (gray bars) at 6 months old indicates sub-clinical levels of ALP and AST in serum as compared to wild type animals (black bars).

FIG. 10 shows a Western blot analysis of wild type and R408W pigs as compared to muscle homogenate as a negative control for PAH. The blot shows that a 54 kDa PAH monomer (mutant/inactive) was detected in all PAH^{R408W/R408W} piglets in amounts similar to that of wild type large white pig liver.

FIG. 11 shows a sequence alignment of humanized pig PAHhR408W (top) showing the guide RNA for the RNPs as well as a homology template modified to revert the mutation and disrupt the PAM to prevent re-cutting. The location of the R408W mutation (red letters) and engineered BsaI site are also indicated.

FIG. 12 shows a T7 endonuclease assay of PAH PCR products derived from hR408W fibroblasts, fibroblasts treated with RNPs containing the PAH gRNA, and a positive control for T7 cutting.

FIG. 13 shows quantitation of amplicon sequences derived from unedited (blue) and RNP treated (red) PCR of the PAHhR408W locus as well as the top 12 predicted off-target cutting sites for the gRNA employed.

FIG. 14 is restriction fragment length polymorphism (RFLP) analysis with a diagram of the position of the BsaI cut site. Positive control was based on a synthesized dsDNA encoding the intended HDR product.

FIG. 15 shows PCR results indicating the presence of the anticipated product in co-transfected cells, but not untreated hR408W fibroblasts.

FIG. 16 shows the relative abundance of R408W and corrected sequences in cells.

FIG. 17 shows a pie chart indicating the relative presence of unedited (R408W), NHEJ, HDR (W408R and BsaI site), and incomplete HDR (not all corrections present) species in PAH locus amplicon sequencing from FIG. 16.

### DETAILED DESCRIPTION

The present disclosure is directed to methods, vectors, and cells for introducing genetic mutations into non-human animal cells. More specifically, the present disclosure is directed to methods of introducing mutations into a gene of interest to modify its function and modifying the sequences flanking the gene of interest to be homologous with human DNA. Nuclei from cells containing these DNA sequences can be used to create transgenic embryos which can be implanted into an adult female animal for gestation. Animals produced by these methods can be used to model human diseases associated with mutated genes. The models are useful for testing human gene therapies because the flanking sequences surrounding the gene of interest match the sequences of human DNA, thus enabling direct testing of gene therapies designed for human subjects in the animal model.

Recombinant vectors and cells are described that include DNA sequences encoding mutations in enzymes that cause metabolic disorders. The sequences flanking the mutations can be modified to be homologous with human DNA. In some embodiments, methods for making vectors, cells, and animals involve the use of targeted nuclease systems such as CRISPR/Cas9, zinc finger nucleases, and TALENs to introduce double-stranded breaks in the DNA of the model animal. Homology-directed repair (HDR) template oligonucleotides are designed to introduce the desired mutation into the DNA of the model animal along with humanized flanking sequences. The humanized flanking sequences facilitate testing of gene editing therapies in the animal model using constructs designed for human therapies. In some embodiments, the animal model is a pig model for an inherited metabolic disorder such as phenylketonuria (PKU).

In some embodiments, a method of introducing a mutation into a gene of interest in a non-human animal cell comprises: designing a targeted nuclease system to introduce double-stranded breaks in the DNA flanking the gene of interest; designing a homology-directed repair (HDR) template oligonucleotide comprising a DNA sequence encoding a mutation in the gene of interest flanked by DNA sequences homologous to human DNA sequences flanking the mutation; and delivering the targeted nuclease system and HDR template oligonucleotide into the non-human animal cell.

In some embodiments, the mutation causes loss of function of an enzyme. In some embodiments, the enzyme is a hydroxylase. In some embodiments, the mutation is a missense mutation. In some embodiments, the missense mutation is commonly associated with a disease caused by reduced or absent enzyme activity. In some embodiments, the disease is a metabolic disorder.

In some embodiments, the non-human animal is a mammal. In some embodiments, the mammal is selected from the group comprising a swine, a bovine, a sheep, a goat, a horse, a deer, a primate, and a dog.

In some embodiments, the targeted nuclease system is selected from the group consisting of zinc finger nucleases, CRISPR/Cas9 endonucleases, and TAL effector nucleases. In some embodiments, the double-stranded breaks occur no more than 50 nucleotides away from the gene of interest. In some embodiments, at least one single nucleotide polymorphism (SNP) modifies the DNA at least 15 nucleotides upstream and downstream of the mutation to match human sequences.

In some embodiments, the cell is a somatic cell. In some embodiments, the targeted nuclease system and HDR template oligonucleotide are delivered into the cell using at least one recombinant vector. In some embodiments, the recombinant vector is a viral vector selected from the group comprising: retroviral vector, lentiviral vector, adenoviral vector, and adeno-associated vector.

In some embodiments, the method further comprises transferring DNA from the cell into an embryo of the non-human animal. In some embodiments, the DNA is transferred from the cell into the embryo using somatic cell nuclear transfer. In some embodiments, the DNA is transferred from the cell into the embryo using chromatin transfer. In some embodiments, the non-human animal is a pig. In some embodiments, the cell is a fibroblast.

In some embodiments, DNA sequences homologous to human DNA sequences flanking the gene of interest are created by comparing the human DNA sequences surrounding the gene of interest with the non-human animal DNA sequences surrounding the gene of interest and introducing single-nucleotide polymorphisms (SNPs) into the DNA sequences of the non-human animal to match the human DNA sequences. In some embodiments, the gene of interest is Pah.

In some embodiments, a transgenic non-human animal cell has a genome comprising a DNA sequence. The DNA sequence includes a mutation in a target gene; and flanking sequences having one or more single nucleotide polymorphisms (SNPs) that cause at least 20 nucleotides upstream and downstream of the mutation to be homologous with human DNA.

In some embodiments, the mutation causes loss of function of an enzyme. In some embodiments, the mutation is a missense mutation commonly associated with a disease caused by reduced or absent enzyme activity. In some embodiments, the enzyme is phenylalanine hydroxylase. In some embodiments, the DNA sequence comprises (5′-TCTCAGATCTCTGGTTTTGGTCTTAGGAACTTTGCTGCCACAATACCTTGGCC CTTCTCAGTTCGCTACGACCCATACACCCAAAGGATT-3′) (SEQ ID 16).

In some embodiments, the non-human animal is an ungulate. In some embodiments, the ungulate is a pig of the species Sus scrofa. In some embodiments, the cell is an embryo. In some embodiments, the cell is a primary somatic cell.

In some embodiments, a recombinant vector comprises a polynucleotide. The polynucleotide encodes: a targeted nuclease system designed to introduce double-stranded breaks in the DNA flanking a gene of interest in a non-human animal; and a homology-directed repair (DR) template having a DNA sequence including a mutation in the gene of interest that modifies its function and flanking sequences mutated to be homologous to human DNA flanking the location of the mutation.

In some embodiments, the mutation causes a reduction or loss of function of the gene of interest. In some embodiments, the gene of interest is a gene associated with an inborn error of metabolism. In some embodiments, the inborn error of metabolism is phenylketonuria.

In some embodiments, the targeted nuclease system is selected from the group comprising zinc finger nucleases, CRISPR/Cas9 endonucleases, and TAL effector nucleases. In some embodiments, the flanking sequences are at least 15 nucleotides long. In some embodiments, the flanking sequences are at least 20 nucleotides long. In some embodiments, the flanking sequences are at least 30 nucleotides long.

In some embodiments, the recombinant vector is a viral vector selected from the group comprising: retroviral vector, lentiviral vector, adenoviral vector, and adeno-associated vector. In some embodiments, the non-human animal is a pig. In some embodiments, the homology-directed repair (HDR) template DNA sequence comprises (5′-TCTCAGATCTCTGGTTTTGGTCTTAGGAACTTTGCTGCCACAATACCTT GGCCCTTCTCAGTTCGCTACGACCCATACACCCAAAGGATT-3′) (SEQ ID 16).

In some embodiments, a non-human mammal model of an inborn error of metabolism has a DNA sequence comprising: a mutation in a gene that causes the inborn error of metabolism, and two or more single nucleotide polymorphisms (SNPs) in the DNA flanking the mutation that causes the DNA to match a homologous human DNA sequence. In some embodiments, the inborn error of metabolism is one of adrenoleukodystrophy, Gaucher disease, hereditary hemochromatosis, Lesch-Nyhan syndrome, Maple syrup urine disease, Glucose galactose malabsorption disease, Menkes syndrome, Niemann-Pick disease, phenylketonuria, Refsum disease, Tangier disease, Tay-Sachs disease, Wilson's disease, Zellweger syndrome, Alkaptonuria, Carnosinemia, Cystinuria, fumaric aciduria, Tyrosinemia, Sarcosinemia, Hyperlysinemia, and Hyperprolinemia. In some embodiments, the non-human mammal is a pig and the inborn error of metabolism is phenylketonuria (PKU). In some embodiments, the DNA sequence comprises (5′-TCTCAGATCTCTGGTTTTGGTCTTAGGAACTTTGCTGCCACAA TACCTTGGCCCTTCTCAGTTCGCTACGACCCATACACCCAAAGGATT-3′) (SEQ ID 16).

In some embodiments, a method of producing a porcine model of phenylketonuria comprises: transferring a nucleus from a donor cell to a denucleated pig oocyte to create a transgenic embryo, the nucleus comprising DNA encoding a Pah gene having a mutation causing loss of function and flanking regions surrounding the mutation that are homologous to human DNA; and implanting the transgenic embryo into a sow.

In some embodiments, the flanking regions are at least 20 nucleotides in length. In some embodiments, the mutation causing loss of function of the Pah gene is caused by a R408W mutation in exon 8 of the pig genome. In some embodiments, the DNA comprises the sequence (5′-TCTCAGATCTCTGGTTTTGGTCTTAGGAACT TTGCTGCCACAATACCTTGGCCCTTCTCAGTTCGCTACGACCCATACACCCAA AGGATT-3′) (SEQ ID 16).

In some embodiments, the method of producing a porcine model of phenylketonuria further comprises generating DNA encoding a mutated Pah gene having a mutation causing loss of function and flanking regions surrounding the mutation that are homologous to human DNA by: designing a targeted nuclease system to introduce double-stranded breaks in the DNA flanking the location of the mutation causing loss of function; designing a homology-directed repair (DR) template oligonucleotide comprising a DNA sequence encoding a R408W mutation in exon 8 flanked by DNA sequences having single nucleotide polymorphisms making the DNA homologous to human DNA sequences; and delivering the targeted nuclease system and HDR donor nucleotide into the donor cell.

### Definitions

The term “DNA” means deoxyribonucleic acid. DNA is a double-stranded polynucleotide encoding instructions for the production of proteins. A DNA sequence refers to the order in which different bases (cytosine, guanine, adenine, thymine) are arranged.

The term “nucleotide” refers to an organic molecule that serves as a subunit of DNA or RNA. The molecule includes a nitrogenous base, a five-carbon sugar, and at least one phosphate group. A string of nucleotides makes up a “nucleic acid” or “polynucleotide.”

The term “gene” refers to a sequence of nucleotides that encode a particular protein. The term “gene of interest” or “target gene” refers to a gene being studied in a model animal by introducing a mutation into the gene.

The term “targeted nuclease system” or “engineered nuclease” refers to endonucleases designed to cut DNA at particular locations based on guidance from at least one targeting peptide. Examples include zinc finger nucleases, TALENs, and CRISPR/Cas9.

The term “double-stranded break” means a cut in both strands of a DNA polynucleotide molecule.

The term “mutation” means an alteration to a nucleotide sequence. The alterations can be caused by one or more nucleotide additions, substitutions or deletions. As used in this specification, “mutation” refers to a change to a nucleotide sequence that results in a change in at least one amino acid that is coded by the nucleotides.

The term “single nucleotide polymorphism (SNP)” means a single nucleotide substitution that occurs at a particular location in a genome.

The term “missense mutation” means a point mutation (single nucleotide polymorphism) that changes which codon is encoded by DNA and thus changes which amino acid will be produced during protein synthesis.

The term “somatic cell” means a non-germline cell.

The term “ungulate” means a hoofed mammal. Examples of ungulates include cattle, deer, horses, and pigs.

The term “inborn error of metabolism” or “congenital metabolic disease” or “inherited metabolic disorder” refers to a disorder caused by defects in one or more genes that encode enzymes involved in metabolism.

### Method Overview

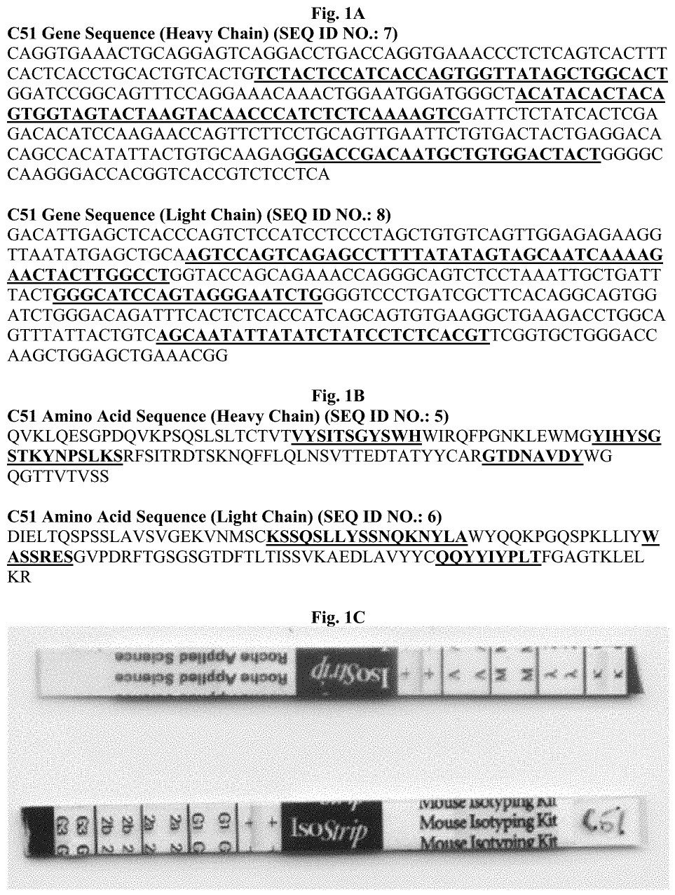

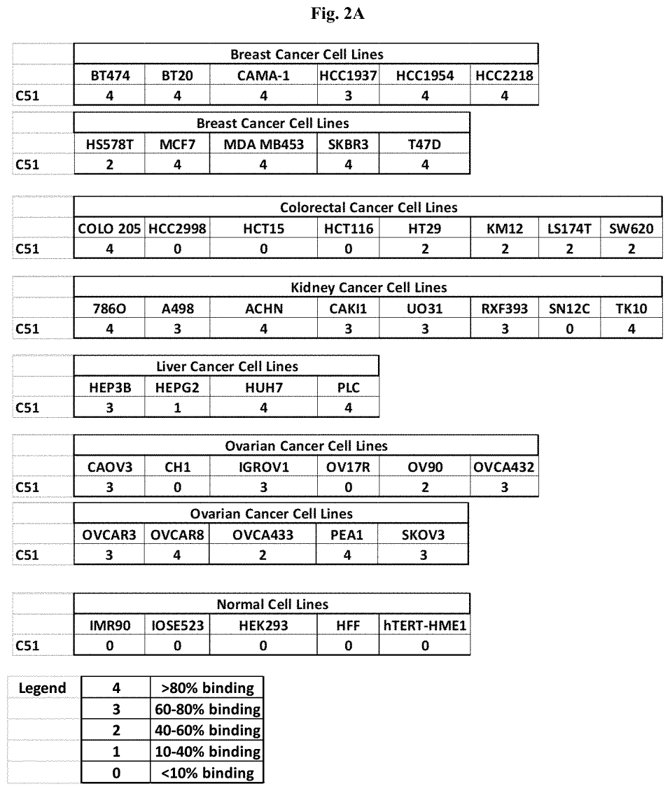

FIG. 1 illustrates a schematic diagram outlining a method of introducing a gene mutation flanked on both sides by humanized DNA sequences into the genome of a non-human animal. The method can be used to produce a vector, which can be used to transfect or transduce a non-human animal cell, which can be used to generate a model animal. The location of a target gene and a desired mutation are identified in a target exon of the genome of the non-human animal. A targeted nuclease system employing zinc finger nucleases, CRISPR/Cas9 endonucleases, or TAL effector nucleases (TALENs) is used to introduce double-stranded breaks upstream and downstream of the targeted gene mutation. A homologous sequence including the target mutation and one or more single nucleotide polymorphisms (SNPs) in the flanking sequences is used to repair the break. The result is a recombinant DNA sequence including non-human animal sequences, the desired mutation in the target gene, and human flanking sequences.

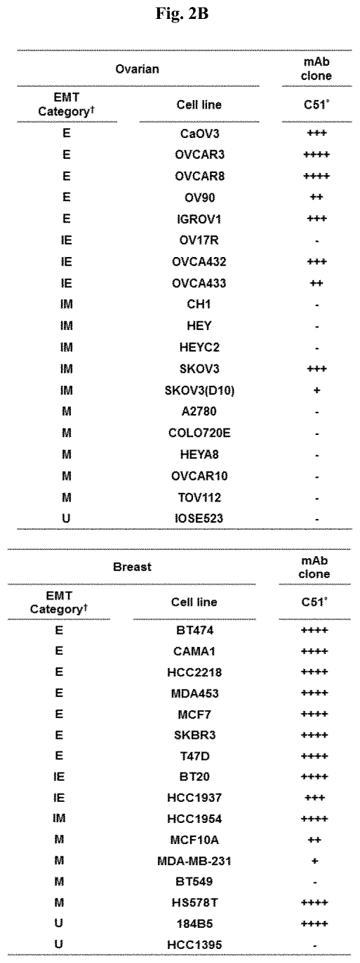

FIG. 2 illustrates a schematic diagram outlining a method of transferring the modified DNA sequence into an embryo to produce a model animal. In the example of FIG. 2, the modified DNA is introduced into a donor cell (fibroblast). The genetic material from the donor cell is transferred into a denucleated oocyte taken from a female pig. The resultant recombinant embryo is implanted into a female pig. The resultant piglets will have the modified DNA sequence including the target gene mutation. Thus, the piglets will serve as models for the desired metabolic disorder.

### Materials and Methods

Animal, Gene, and Mutation Selection

Many human diseases are caused by genetic mutations that inactivate or reduce the activity of a particular enzyme. Those diseases that result from mutations in genes that exist at birth can be referred to as “inborn errors of metabolism,” “congenital metabolic diseases,” or “inherited metabolic disorders.” Examples of inborn errors of metabolism include adrenoleukodystrophy, Gaucher disease, hereditary hemochromatosis, Lesch-Nyhan syndrome, Maple syrup urine disease, Glucose galactose malabsorption disease, Menkes syndrome, Niemann-Pick disease, phenylketonuria, Refsum disease, Tangier disease, Tay-Sachs disease, Wilson's disease, Zellweger syndrome, Alkaptonuria, Carnosinemia, Cystinuria, fumaric aciduria, Tyrosinemia, Sarcosinemia, Hyperlysinemia, and Hyperprolinemia. In some embodiments, the inborn error of metabolism is phenylketonuria (PKU).

For the disorders where a particular gene has been identified for being linked to the disease phenotype, animals having the equivalent or homologous genetic mutation can serve as useful models for the human disease. Various types of mutations can lead to disruption of enzyme activity, including truncations, deletions, missense mutations, frameshift mutations, and insertions. In some instances, a mutation can be specific to a single nucleotide or amino acid. In some embodiments, the mutation selected for use in an animal model is homologous to a mutation that causes a human metabolic disorder. The location of the homologous nucleotide or amino acid needs to be identified in the model animal. For that reason, it must be confirmed that the model animal has a homologous gene. This can be accomplished using online software tools such as NCBI's HomoloGene database. In some embodiments, the target gene is Pah and the mutation is R408W in exon 8 of the pig genome, which is equivalent to the Pah gene in exon 12 in humans.

Animals that can serve as models for human diseases can be any vertebrate non-human animal. In some embodiments, the non-human animal is a mammal. The mammal can be selected from ungulates such as swine, bovine, sheep, goats, horses, and deer. In some embodiments, the animal is a pig. Typically the pigs used for research models are domesticated pigs having the species Sus scrofa. Examples of breeds of pigs that can be used in research include Landrace, Large white, Duroc, Gottingen, and Ossabaw.

Designing the Nuclease System

Engineered nucleases or targeted nucleases combine a non-specific nuclease enzyme (“molecular scissors”) with one or more DNA sequence recognizing peptides that target specific, short (1-18) nucleotide sequences. The sequence recognizing peptides are selected to target specific locations within a DNA sequence to cleave with a double-stranded break. The targeted nuclease system could utilize zinc finger nucleases (ZFNs), transcription activator-like effector nucleases (TALENs), or clustered regularly interspaced short palindromic repeats (CRISPR) with CRISPR associated proteins (Cas).

A combination of endonuclease and one or more DNA sequence recognizing peptides are designed to target the particular locations within the genome where cleavage is desired. In some embodiments, the double-stranded breaks are designed to be at least 15 nucleotides upstream and downstream of the desired target gene mutation. In some embodiments, the double-stranded breaks are at least 20 nucleotides, at least 25 nucleotides, or at least 30 nucleotides upstream and downstream of the target gene mutation. In some embodiments, the double-stranded breaks are no more than 50 nucleotides upstream or downstream of the target gene mutation. The nucleotide sequence encoding the endonuclease system is generated. In some embodiments, software tools and other services can be employed to more easily identify nuclease system components to target particular locations for cleavage.

Designing the HDR Template

A homology-directed repair (HDR) template is designed to insert the desired gene mutation into the DNA that is cleaved by the nuclease system. In addition to the nucleotides encoding the target mutation, flanking sequences are designed to replace the nucleotides that are cut from the target DNA. The flanking sequences of the non-human animal DNA are compared with the homologous flanking sequences of a human. The flanking sequences in the HDR template are modified with one or more SNPs to match the human flanking sequences. The HDR template thus includes “humanized” sequences that will allow for gene editing systems designed for human treatment to recognize the DNA sequences flanking either sides of the target mutation. These gene editing systems can includes ZFNs, TALENs, and CRISPR/Cas systems that are designed to modify human DNA. The gene editing systems could therefore be tested in the non-human animal without modification because the sequences flanking both sides of the gene mutation are modified to match the human flanking sequences.

Introducing Humanized DNA Constructs into Cells

The HDR template and endonuclease system nucleotide sequences are inserted into a viral vector. The viral vector can be selected from retroviral vectors, lentiviral vectors, adenoviral vectors, and adeno-associated vectors. In some embodiments, the viral vector is preferably an adeno-associated vector (AAV). The viral vector includes sequences encoding packaging proteins needed for the virus to introduce the HDR template and endonuclease system DNA into non-animal cells.

After the virus has been produced used the vector, it is used to transduce cells. The cells can be somatic cells or germ cells. In some embodiments, the cells are primary somatic cells. The donor somatic cells could be selected from epithelial cells, fibroblast cells, cumulus cells, granulosa cells, and luteal cells. In some embodiments, the cells are embryos.

For techniques where the vectors are introduced to somatic cells, the DNA material needs to be transferred into an embryo. Somatic cell nuclear transfer is a common method used in cloning. A mature oocyte is isolated and its nucleus is removed. The nucleus of the somatic donor cell is inserted into the denucleated oocyte to form a recombinant embryo.

Another technique for transferring genetic material between cells is chromatin transfer. Chromatin within the donor cell is remodeled to remove somatic regulatory proteins. Then the somatic cell is fused to an oocyte to produce a recombinant embryo.

Implantation and Gestation

Recombinant embryos are implanted into adult female animals for gestation. In some embodiments, the oocytes are from pigs and the recombinant embryos are implanted into the wombs of sows. The resultant young have genomes including the target gene mutation.

Various characterization procedures can be performed to confirm that the recombinant model animals have the desired DNA sequences and phenotypes. In the example of Pah pigs, the amino acid levels in the pig indicate whether there is normal PAH activity or not.

### EXAMPLES

### Example 1

Phenylketonuria (PKU) is a metabolic disorder whereby phenylalanine metabolism is deficient due to allelic variations in the gene for phenylalanine hydroxylase (PAH). There is no cure for PKU other than orthotopic liver transplantation, and the standard of care for patients is limited to dietary restrictions and key amino acid supplementation. Therefore, Pah was targeted in pig fibroblasts using TALENs, and pigs were subsequently cloned to facilitate research and therapeutic development where the genetic variation is identical to a common and severe human allele, R408W. Additionally, the proximal region to the mutation was further humanized by introducing 5 single nucleotide polymorphisms (SNPs) to allow for development of gene editing machinery that could be translated from the pig directly to human PKU patients that harbor at least one classic R408W allele.

### Design of TALENs and Repair Sequences

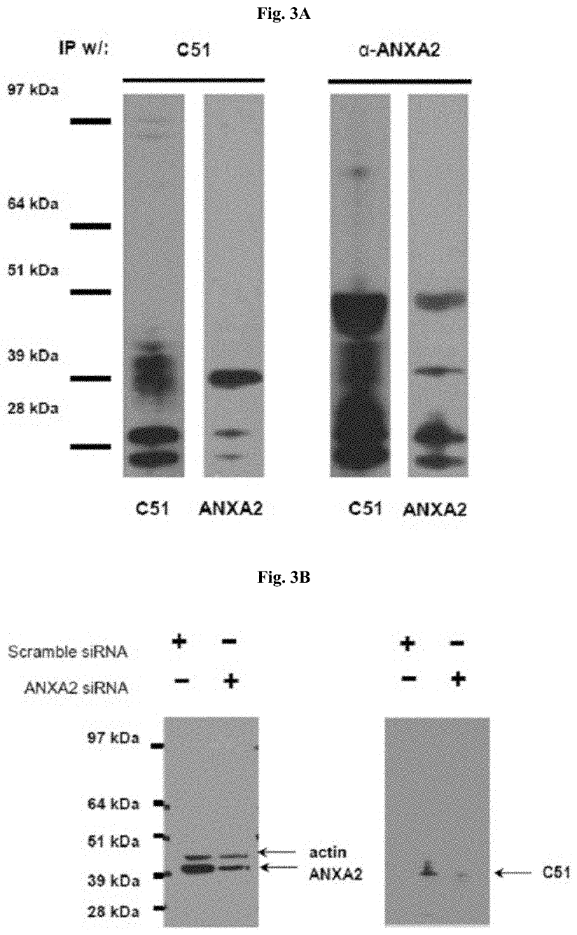

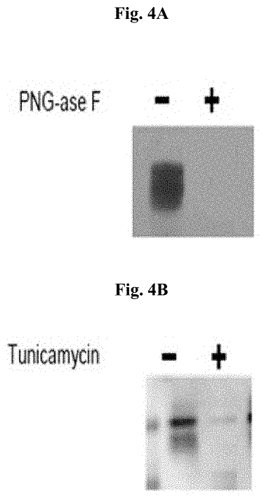

To simulate the effects of PKU, the PAH gene was targeted for mutation in pig fibroblasts. R408W in exon 8 was previously identified as a mutation that would cause loss of function of phenylalanine hydroxylase in pigs. The location of the Pah allele in pig fibroblasts is diagrammed in FIG. 3 (SEQ IDs 1-3). TALENs were designed to target Exon 8 of Sus scrofa (ss) PAH. The location of the right monomer was strategically placed to contain a mismatch following a successful R408W HDR event. Additionally, the sequences upstream and downstream of the R408W mutation were compared between exon 8 in wild-type pigs (ssPAH) (SEQ IDs 6-7) and the homologous region in human exon 12 (hsPAH) (SEQ IDs 4-5) to identify any differences (see FIG. 4). Sequence alignment of ssPAH and hsPAH shows the high similarity surrounding the target R408W mutation. 5 sites were identified for mutation with single nucleotide polymorphisms (SNPs) to “humanize” the sequences surrounding the PAH mutation. FIG. 4 illustrates the homology template sequence (HT) (SEQ IDs 8-9) that was designed to guide repair of the cleaved DNA to include the R408W mutation as well as 5 SNPs that allow for targeting with human-translatable gene editing reagents. The PAH KO is also shown with SEQ IDs 10-11. Homology template sequences can be synthesized by well-known methods-many of which can be performed by vendors such as Integrated DNA Technologies (IDT, Coralville, Iowa).

Candidate TALEN target DNA sequences and repeat variable diresidue (RVD) sequences were identified using the online tool “TAL EFFECTOR NUCLEOTIDE TARGETER 2.0”.

Sequences for homology-directed repair (HDR) were designed to integrate the mutations discussed above. A single-stranded donor oligonucleotide (ssODN) was designed having the sequence (5′-TCTCAGATCTCTGGTTTTGGTCTTAGGA ACTTTGCTGCCACAATACCTTGGCCCTTCTCAGTTCGCTACGACCCATACACC CAAAGGATT-3′) (SEQ ID 16).

### TALEN Production

Plasmids for in vitro TALEN mRNA transcription were then constructed by following the Golden Gate Assembly protocol using RCIscript-GOLDYTALEN (Addgene ID 38143) as final destination vector (Carlson, 2012). Assembled RCIscript vectors prepared using the QIAPREP SPIN MINIPREP kit (Qiagen) were linearized by SacI (NEB) to be used as templates for in vitro TALEN mRNA transcription using the mMESSAGE mMACHINE® T3 Kit (Ambion) (Carlson, 2009). Resulting mRNA was DNase treated prior to purification using the RNeasy Kit (Qiagen).

### Tissue Culture and Transfection

Outbred Ossabaw and large white pig fibroblasts were briefly maintained at 38.5° C. at 5% CO₂ in DMEM supplemented with 10% fetal bovine serum, 100 I.U./mL penicillin and streptomycin, 2 mM L-Glutamine and 10 mM Hepes. Once fibroblasts reached 90% confluency, they were spilt 1:2 and harvested the next day. The Neon Transfection system (Life Technologies) was used to deliver the TALEN mRNA (500 ng each; ssPAH 8.1 L, ssPAH 8.1 R) and ssODN (0.2 nmoles; ssPAH R-W 90 (5′-TCTCAGATCTCTGGTTTTGGTCTTAGGAACTTTGCTGCCACAATACCTTGGCC CTTCTCAGTTCGCTACGACCCATACACCCAAAGGATT-3′)) (SEQ ID 16). Approximately 600,000 cells were resuspended in “R” Buffer with mRNA TALENs and HDR oligo, and electroporated using the 100 μL tips and the following parameters: input voltage: 1800V; pulse width: 20 ms; pulse number: 1. Transfected cells were dispersed into one well of a 6-well plate with 2 mL DMEM media and cultured for 3 days at 30° C. prior to population efficiency testing.

### Sample Preparation

Transfected cell populations were collected. 50% of the cells were re-seeded onto one well of a 6-well plate with 2 mL fresh DMEM growth media, 40% were resuspended in 80 μL cryopreservation media (90% FBS, 10% DMSO), and 10% were resuspended in 20 μL of 1×PCR compatible lysis buffer (10 mM Tris-Cl pH 8.0, 2 mM EDTA, 0.45% Tryton X-100 (vol/vol), 0.45% Tween-20 (vol/vol)) freshly supplemented with 200 μg/ml Proteinase K. The lysates were incubated in a thermal cycler using the following program: 55° C. for 60 minutes, 95° C. for 15 minutes.

### TALEN Efficiency

PCR amplification was conducted using AccuStart™ Taq DNA Polymerase HiFi (Quanta Biosciences) with 1 μL of the cell lysate as template. The following primers and program were used: ssPAH E8 F1 (5′-CTTCACCTCTCAGCCTGGTC-3′) (SEQ ID 17) and ssPAHE8 R1 (5′-TGCCACGTTTCGTTCTCTCA-3′) (SEQ ID 18); 1 cycle (95° C., 2 minutes), 35 cycles of (95° C., 20 s; 62° C., 20 s; 68° C., 45 s), 1 cycle (68° C., 5 minutes). Frequency of mutations in the population was analyzed with the SURVEYOR MUTATION DETECTION Kit (Transgenomic) according to the manufacturer's recommendations, using 10 μL of the PCR product. The products were resolved on 10% TBE polyacrylamide gels and visualized by ethidium bromide staining. Densitometry measurements of the bands were performed using ImageJ. Mutation rates of SURVEYOR reactions were calculated.

### Single-Cell Derived Clonal Isolation and Chromatin Transfer

Four days post transfection, cells were seeded onto 10 cm plates at a density of 100 cells/plate and cultured until individual colonies reached approximately 5 mm in diameter. Growth media was aspirated and the plates were washed with 4 mL PBS. 8 mL of a 1:4 (vol/vol) mixture of TrypLE and DMEM was added and colonies were aspirated in a volume of 150 μL, transferred into wells of a 48-well plate containing 150 μL DMEM growth media, and mixed via manual pipetting. 150 μL of the mixture was seeded into a replica 96-well plate and cultured at 38.5° C. The 96-well plates were incubated for 2 days prior to lysis (described above). PCR amplification using AccuStart™ II PCR SuperMix (Quanta Biosciences) was performed. Amplicons were purified using the QIAquick 96 PCR Purification Kit (QIAGEN) following manufacturer's instructions and submitted for Sanger sequencing (ACGT, Inc.). Clones containing the desired genotype were cryopreserved in 70 μL cryopreservation media and submitted to Cooperative Resources International Center for Biotechnology for Chromatin Transfer.

### Piglets

After clonal selection and growth of a homozygous pig fibroblast, R408W piglets were created by somatic cell nuclear transfer (SCNT) into Ossabaw or 50% large white/50% landrace farm pigs as previously described (Carlson, 2012). All piglets cloned by this process (Ossabaw or large white/landrace) were verified to be homozygous for the R408W mutation as well as the humanizing SNPs, as represented by Piglet No. 1769 (FIG. 2, PAH KO).

All procedures involving live animals were conducted in compliance with regulations outlined by the Institutional Animal Care and Use Committees of Cooperative Resources International (CRI) International Center for Biotechnology (IBC) and Mayo Clinic. The piglets were hand-reared on a combination of commercially available bovine colostrum/milk replacers (Bovine IgG Calf's Choice Total®Gold, SCCL, SK, Canada; CL Sow Replacer, Cuprem®, Kenesaw, Nebr., USA; Birthright™, Ralco Animal Nutrition, Marshall, Minn., USA) and a phenylalanine-free human infant formula (Phenex®-1, Abbot Nutrition, IL, USA).

Five total pregnancies resulted in eight live born PAI^{R408W/R408W} piglets delivered via cesarean section on day 118 of gestation. A summary of the piglets are provided in Table 1.

TABLE 1
Genotypes of all PAH-targeted piglets

| Pig ID | Breed | Genotype |
|---|---|---|
| Pregnancy A | | |
| 1769 | Ossabaw | hR408W Homozygote |
| Pregnancy B | Yorkshire | hR408W Homozygote |
| 1794 | Yorkshire | hR408W Homozygote |
| 1795 | Yorkshire | hR408W Homozygote |
| 1796 | Yorkshire | hR408W Homozygote |
| 1797 | Yorkshire | hR408W Homozygote |
| Pregnancy C | | |
| 1798 | Yorkshire | hR408W Homozygote |
| Pregnancy D (NTBC) | | |
| 21 (still born) | Yorkshire | hR408W Homozygote |
| 22 Charm | Yorkshire | Compound Heterozygote-hR408W; |
| | | pA403GfsX47¹ |
| 23 Lucky | Yorkshire | Compound Heterozygote-hR408W; |
| | | pA403GfsX47¹ |
| Pregnancy E (NTBC) | | |
| 899 Cornflake | Yorkshire | hR408W/R408W compound |
| | | heterozygote-one allele |
| | | lacks the two 5′ humanized bases² |
| 900 Cheerio | Yorkshire | hR408W/R408W compound |
| | | heterozygote-one allele |
| | | lacks the two 5′ humanized bases² |

¹A frame-shifting indel introduces a stop codon 47 amino acids downstream in A403GfsX47
²Incompletely humanized R408W

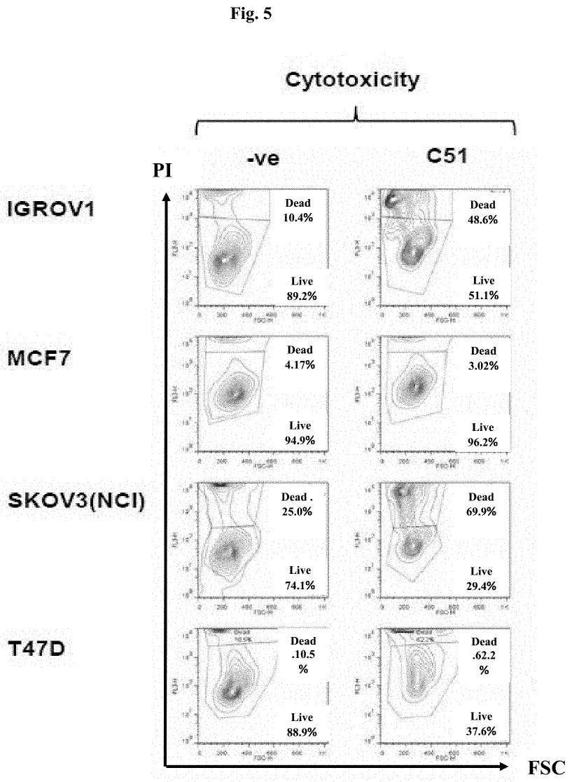

This was initially performed in the Ossabaw background, which resulted in a single piglet (Pregnancy A). This piglet was below the average weight for Ossabaw piglets (FIG. 5, 464 gvs 650 g) and demonstrated marked hypopigmentation characteristic of disrupted phenylalanine metabolism. Plasma from cord blood showed elevated phenylalanine at birth (247 μM compared to 70 μM for the wild type sow).

The piglet was fed colostrum replacer for the first 24 hours, at which point, neurological dysfunction developed, manifesting as lethargy, poor feeding, and ataxia. A peripheral blood sample taken at 24 hours showed arise in plasma phenylalanine to 937 μM. Treatment with Phenex-1 (Abbott Laboratories, Abbott Park, Ill.), a modified amino acid/low-Phe milk replacer used for human PKU patients at 20 kcal/oz was initiated at 24 hours after birth, and within the next 6 hours the piglet showed increased activity levels and normalization of neurologic function. Treatment with Phenex-1 continued until 80 hours old, when the piglet developed scours and ultimately died from complications of failure to thrive. Postmortem plasma analysis showed phenylalanine levels had dropped to 149.8 μM, suggesting that treatment with Phenex-1 was able to normalize blood phenylalanine.

Subsequent SCNT efforts were transitioned to the large white/landrace background (Yorkshire), which is heartier, more familiar to the husbandry groups involved, and less metabolically diverse than the Ossabaw background. This resulted in 9 live piglets in 4 pregnancies (4, 1, 2, and 2, respectively).

The four piglets from the first of these pregnancies (Pregnancy B) were approximately 75% of historical wild type body weight, as shown in FIG. 5. Otherwise the piglets were phenotypically unremarkable at birth. Piglets 1795 and 1796 were robust at birth and independently fed well on colostrum replacer until 36 hours, at which point, severe neurological dysfunction (pedaling, ataxia, epilepsy) was observed and both piglets succumbed to lethal seizures around 40 hours of age. It was unknown if the seizures were related to the metabolic phenotype or possible dehydration from nutritional diarrhea. Piglet 1794, similar to 1769, started showing neurological symptoms (ataxia, lethargy, walking in circles) after consuming colostrum replacer for 24 hours. Treatment with Phenex-1 ameliorated neurological symptoms within four hours, but nutritional diarrhea ultimately led to the death of this piglet around 100 hours of age. Piglet 1797 consumed colostrum replacer for the first 24 hours before treatment with Phenex-1 began. This piglet never fed well independently and ultimately died due to complications of failure to thrive. Post mortem plasma analysis showed elevated phenylalanine in Piglets Nos. 1794-1797 of 349, 341, 274, and 477 μM, respectively.

The Pregnancy C resulted in a single live piglet (No. 1798). This animal had the expected elevated cord blood phenylalanine at 345 μM. With the hypothesis that earlier treatment with Phenex-1 would prevent the onset of neurologic dysfunction and reduce the occurrence of nutritional diarrhea present in the previous litters, Piglet No. 1798 was fed a mixture of colostrum replacer and Phenex-1 at a ratio of 9:1 for the first six hours of life, 3:1 for hours six through 18, 1:1 for hours 18 through 24, and Phenex-1 alone from 24 hours on. No neurological dysfunction was observed, but the onset of mild diarrhea began around 24 hours of age and progressively worsened despite IP fluid therapy and treatment with antibiotics. Ultimately, death occurred around 105 hours. Interestingly, postmortem plasma phenylalanine was within normal limits at 133 μM, suggesting 1) early treatment with Phenex-1 was able to normalize blood phenylalanine and 2) the lethality observed through this pregnancy was not likely related to the phenotype of hyperphenylalaninemia.

For pregnancies D and C, the sows were maintained on NTBC (2-(2-nitro-4-trifluoromethylbenzoyl)-1,3-cyclohexanedione) at a daily dose of 25 mg with the hypothesis that any gestational effect of PAH deficiency could cause hypotyrosinemia, possibly contributing to the underdevelopment of piglets present at birth. By blocking tyrosine metabolism, piglets would be able to better maintain gestational tyrosine levels to ensure availability of this amino acid for in utero growth and development. These PAH KO piglets were analyzed at birth for circulating phenylalanine and tyrosine levels, compared to the sow (assayed at C-section). Wild type tyrosine level is represented for comparison due to the elevated levels present in the sow, as maintained on NTBC.

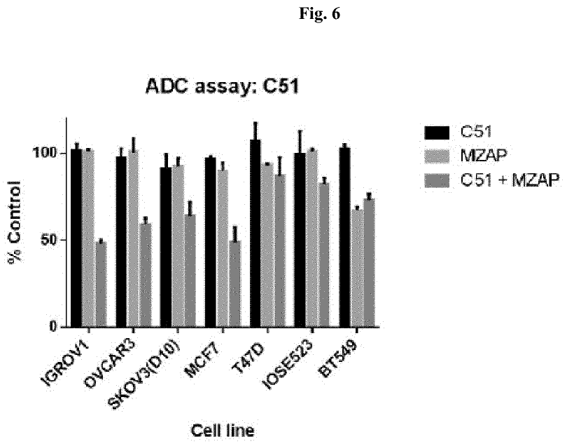

The four live piglets resulting from these final pregnancies were phenotypically unremarkable at birth, with body weights from 1120 to 1630 grams (FIG. 5, NTBC), and phenylalanine levels of 97 to 214 μM (FIG. 6). Surviving piglets in Pregnancies D and E were compound heterozygotes featuring one fully targeted hR408W allele, as well as a second PAH-null allele variety. In Pregnancy D this was a frame-shifted indel that introduced a stop codon 47 amino acids downstream (PAH^{hR408w/A403Gfsx47}) In Pregnancy E this was a partially humanized R408W, where the first two 5′ SNPs were not humanized (PAH^{hR4408Q/R408W}).

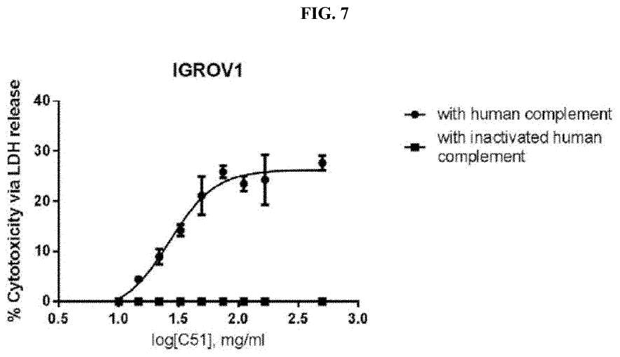

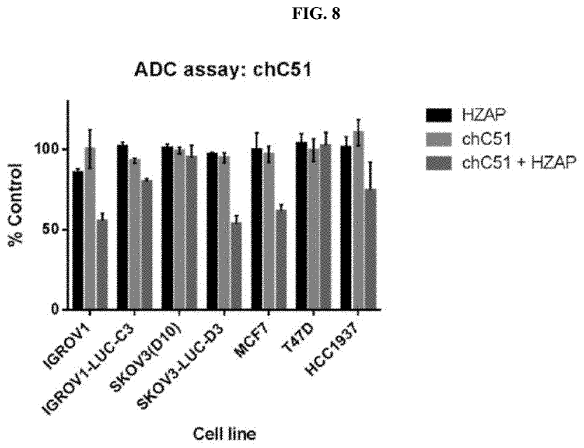

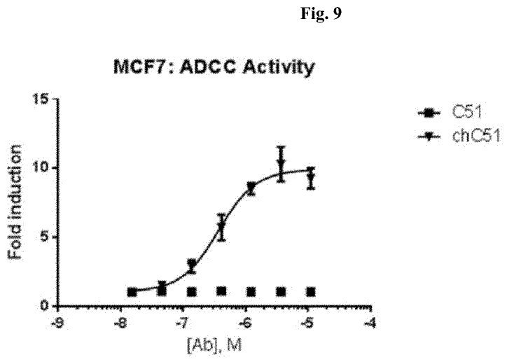

The two live-born piglets were maintained for 6 days (Charm) or continuously (Lucky) to characterize the R408W metabolic phenotype. Serum phenylalanine levels (red) show fluctuation responsive to dietary phenylalanine available (green) in FIG. 8, indicative of disrupted phenylalanine metabolism and modeling the human disease. Tyrosine levels (blue) were elevated at birth due to NTBC administration during gestation, and showed muted responsiveness to phenylalanine levels during development in FIG. 7. The asterisk in the graphs for Lucky represents timing of clinical chemistry analysis in FIG. 9.

One piglet from each litter (22 and 900, subsequently named Charm and Cheerio) had a similar demise as previous litters and died on day 6 of life, but another piglet from each litter (23 and 899, subsequently named Lucky and Cornflake) survived the neonatal period and were maintained for chronic phenotypic characterization.

These piglets all showed elevated tyrosine at birth due to gestational NTBC administration (FIG. 6), which declined to normal levels after birth since the piglets were not maintained on NTBC (FIG. 7). Diet was modified dynamically during the neonatal period, aimed at addressing transient diarrhea while also providing sufficient nutrition in the context of metabolic disease. Therefore, both animals were fed sow colostrum (200 ml each) for the first 7 hours, Phenex-1 starting at hour 8 until day 3, 50% Phenex-1/50% Birthright milk replacer on days 3-6, and 100% Birthright milk replacer from day 6 until week 3 of life. Lucky showed increasing Phe levels until dietary Phe intake was restricted at 3 weeks of age, and the diet was continually modified thereafter to target Phe levels of 100-300 μM. Diet for Cornflake and Cheerio was not modified, and both piglets received normal colostrum replacer for up to 48 hours, pig milk replacer until weaned, and then normal feed. Cornflake showed chronically elevated phenylalanine levels, with fluctuations likely related to growth needs and proximity of blood collections with food consumption. Good association was shown between dietary Phe intake and circulating Phe levels (FIG. 6) consistent with absent PAH activity and consequences of the human PKU phenotype.

Analysis of liver enzymes indicated overall good liver health in the long-term piglet. FIG. 9 shows the serum alkaline phosphatase (ALP) and aspartate aminotransferase (AST) levels in serum of Lucky and Cornflake (gray) at 6 months of age compared to a wild type animal (black). The piglets' ALP and AST levels were below wild type historical control values. However, the analysis showed healthy ALP and AST levels while maintained on a Phe-restricted diet.

### Biochemical Analyses

Post mortem analyses of these piglets included further phenotypic characterization, including PAH expression and activity in the liver as well as brain amino acid constituents. Biochemical assays were used to confirm that the desired mutations were successfully introduced into the piglets.

The piglets were genotyped to confirm that the R408W and 5 humanizing SNPs were introduced in their genomes. FIG. 4 shows a representative piglet chromatogram (PAH KO).

### Western Blot Analysis

Western blotting was performed using an SDS-PAGE electrophoresis system. Homogenized liver samples were quantitated via Bradford assay, and 30-ug aliquots were resuspended in a reducing sample buffer, boiled and run on an 8% acrylamide reducing gel. Gels were blotted to PVDF membrane, and probed with PAH R400 polyclonal antibody (Bioworld Technology, #BS3704) at a dilution of 1:500. GAPDH (ThermoFisher #MA5-15738) was used as a loading control at a dilution of 1:50,000. Two secondary antibodies were used: an HRP-conjugated goat anti-rabbit antibody (Life Technologies #G21234) to bind to the PAH primary, and a goat anti-mouse antibody (Santa Cruz #sc-2055) for the GAPDH. Results were visualized on autoradiograph film using enhanced chemiluminenscence (SuperSignal West Pico Chemiluminescent).

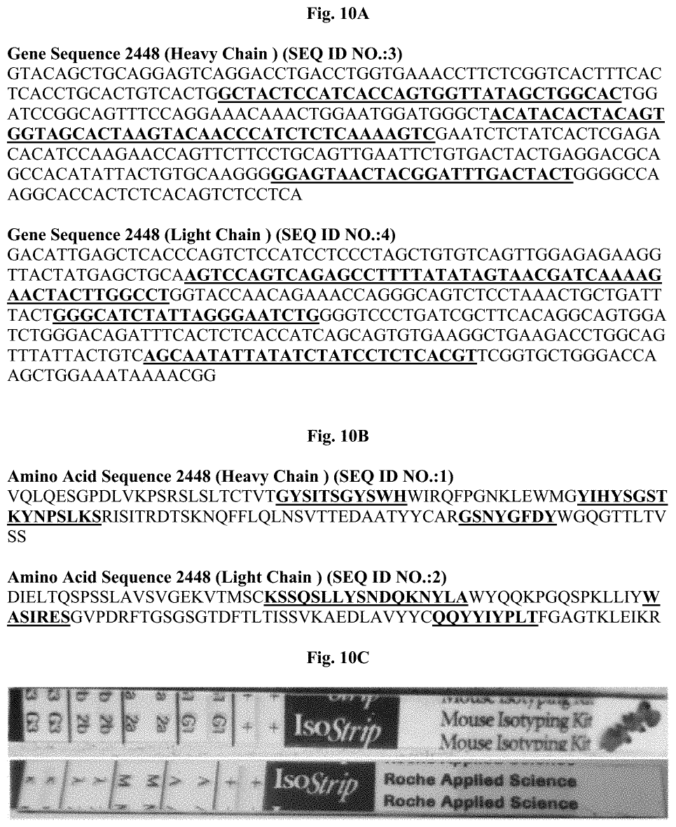

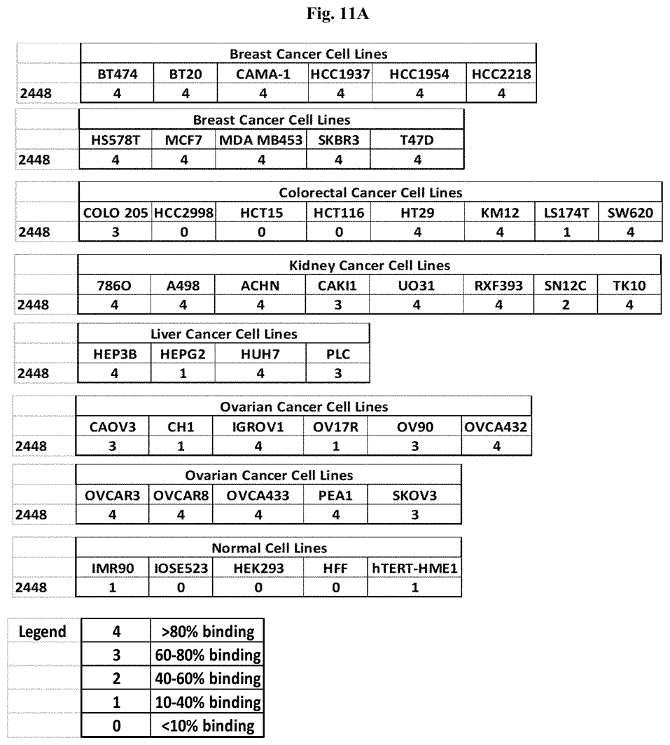

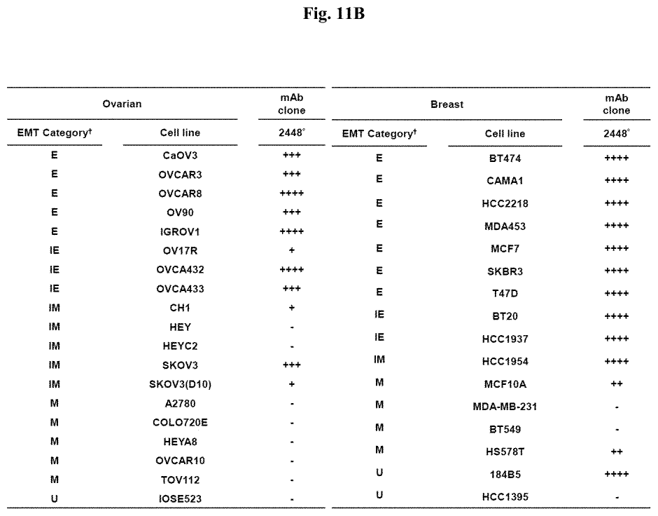

Western blot analysis shows that a 54 kDa PAH monomer (mutant/inactive) was detected in all PAH^{R408W/R408W} piglets in amounts similar to that of wild type large white pig liver. The results shown in FIG. 10 indicate that the mutant protein is still expressed, as is the case in human patients. Muscle homogenate is presented as a negative control for PAH.

### PAH Enzyme Activity Assay

PAH activity was measured in duplicate on liver homogenates of animals harvested within 1 week of birth. A modified radiochemical technique was used for the assay. Briefly, total protein was measured using a bicinchoninic acid procedure (Microprotein Assay; Pierce, Rockford, Ill., USA). Liver homogenates isolated from wild-type C57BL/6 mice were used as positive controls. PAH activity is indicated by the presence of tyrosine (Tyr), which is the product of hydroxylation by PAH of phenylalanine.

Indeed, PAH enzymatic activity was below the level of detection for all PAH^{R408W/R408W} piglets tested, confirming that the R408W mutation was able to achieve complete PAH deficiency despite mutant protein expression (FIG. 9). In other words, a loss of function was achieved with the R408W mutation (* p<0.05).

Amino acid analysis of lysates of brain cortex from PAH^{R408W/R408W} piglets and from wild type controls (Table 2) showed higher levels of phenylalanine in the brain of PAH^{R408W/R408W} piglets than wild type controls. Additionally, PAH^{R408W/R408W} piglets that consumed colostrum replacer alone had higher levels of brain phenylalanine than piglets that were fed Phenex-1, further demonstrating the translatability of this model to the human disease.

TABLE 2

| | Brain amino acid profiles in PAH^{R408W/R408W} piglets | | | | | | | | |
|---|---|---|---|---|---|---|---|---|---|
| | | WT | Normal Diet | | | Treated (Phenex 1) | | | |
| | | Ref | 1795 | 1796 | Mean ± SD | 1794 | 1797 | 1798 | Mean ± SD |
| Amino acid | Age (hrs) | N/A | 36 | 36 | N/A | 100 | 60 | 80 | N/A |
| (nmol/g | Phenyl | 221 | 766.80 | 538.62 | 653 ± 114 | 484.03 | 968.96 | 178.74 | 544 ± 325 |
| wet weight) | alanine | | | | | | | | |
| | Tyrosine | 271 | 207.37 | 193.28 | 200 ± 7 | 384.71 | 281.07 | 210.25 | 292 ± 72 |
| | Tryptophan | 25 | 30.05 | 17.35 | 23.7 ± 6.3 | 94.97 | 50.77 | 41.70 | 62.5 ± 23.3 |
| | Serine | 1355 | 872.48 | 694.33 | 783 ± 89 | 1682.93 | 1461.04 | 1130.67 | 1425 ± 227 |
| | Alanine | 2188 | 508.73 | 660.20 | 584 ± 76 | 3002.46 | 1037.55 | 1450.54 | 1830 ± 846 |
| | Proline | 360 | 281.79 | 190.12 | 236 ± 46 | 1127.43 | 522.59 | 412.54 | 688 ± 314 |

| | | | | | C57B1 | Pah^{enu2/enu2} |
|---|---|---|---|---|---|---|
| | | NTBC | | | Mouse | Mouse |
| | | 21 | 23 | Mean ± SD | Mean ± SD | Mean ± SD |
| Amino acid | Age (hrs) | 168 | 0 | | N/A | N/A |
| (nmol/g | Phenyl | 550 | 350 | 450 ± 100 | 121 ± 66 | 771 ± 80 |
| wet weight) | alanine | | | | | |
| | Tyrosine | 1989 | 668 | 1329 ± 660 | 73 ± 52 | 49 ± 40 |
| | Tryptophan | 36 | 43 | 40 ± 3 | 21 ± 7.9 | 16 ± 7 |
| | Serine | 2421 | 1915 | 2168 ± 253 | 1049 ± 89 | 1297 ± 73 |
| | Alanine | 6067 | 2784 | 4426 ± 1642 | 883 ± 160 | 829 ± 127 |
| | Proline | 1105 | 404 | 755 ± 351 | 216 ± 17 | 233 ± 12 |

### Immunohistological Analysis

Histologically, there were no variations in liver morphology (H&E), fibrosis (Masson's trichrome), or PAH expression patterns (IHC) in PAH KO or wild type livers of mice or pigs (images not shown).

### Homology Template

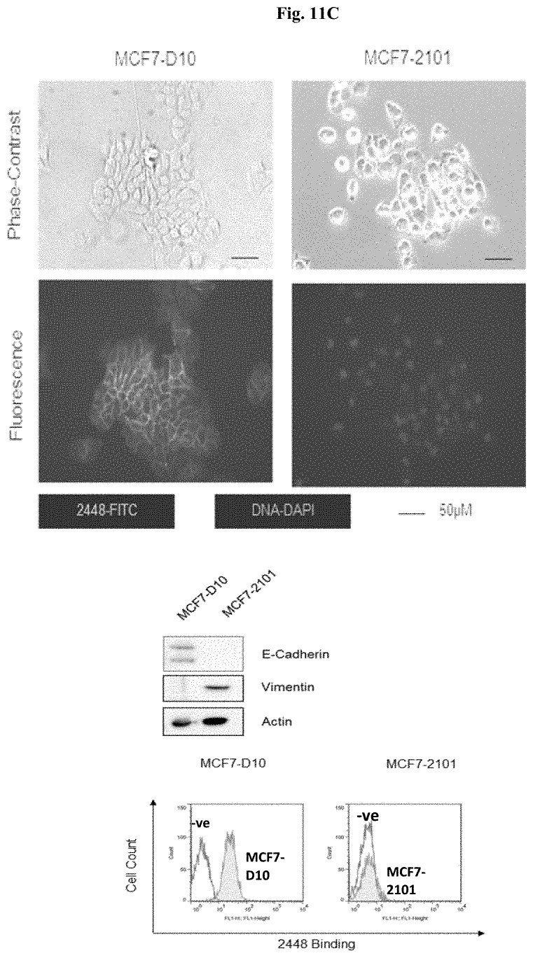

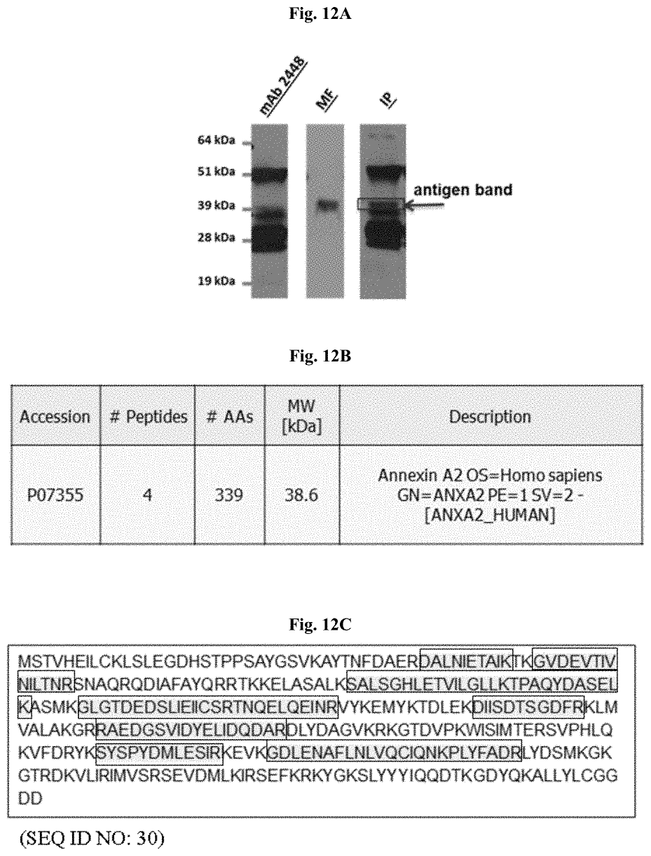

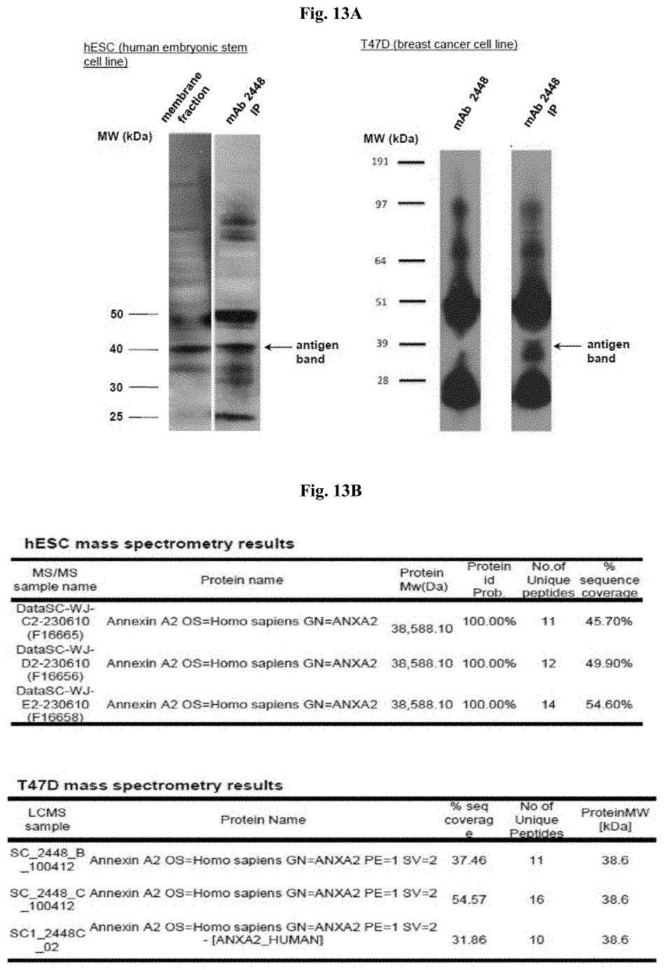

The utility of this PKU model is enhanced by the ability to preclinically develop sequence specific gene editing tools capable of being directly translated to human patients, including those with a classic R408W allele. To that end, a homology template (HT) to correct hR408W (FIG. 11) and associated Cas9 guides were designed to repair the mutant allele as proof of concept toward developing a potential therapeutic gene editing approach. This repair template corresponds to SEQ IDs 14-15 and the humanized pig hR408W allele corresponds to SEQ IDs 12-13. Cas9 ribonucleoprotein complexes (RNPs) containing the R408W-targeting guide RNA were transfected into hR408W fibroblasts and assayed by T7 endonuclease for the introduction of indels. These RNPs induced indel formation at the PAH locus (FIGS. 12 and 13), while analysis of the top 12 predicted off target gRNA binding sites showed no substantial evidence of cutting by T7 (not shown) or amplicon sequencing relative to variation present in untreated controls (FIG. 4c). The top 10 disruptions at PAH relative to the R408W mutation mostly included deletions of 1-14 bp. The unedited sequence was only the second most prevalent detected, indicating very efficient cutting and indel formation.

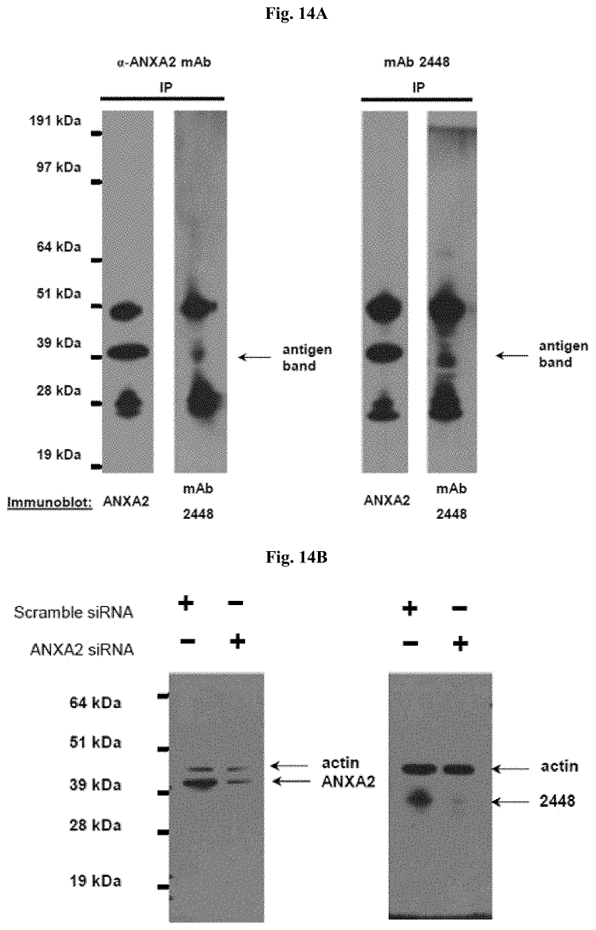

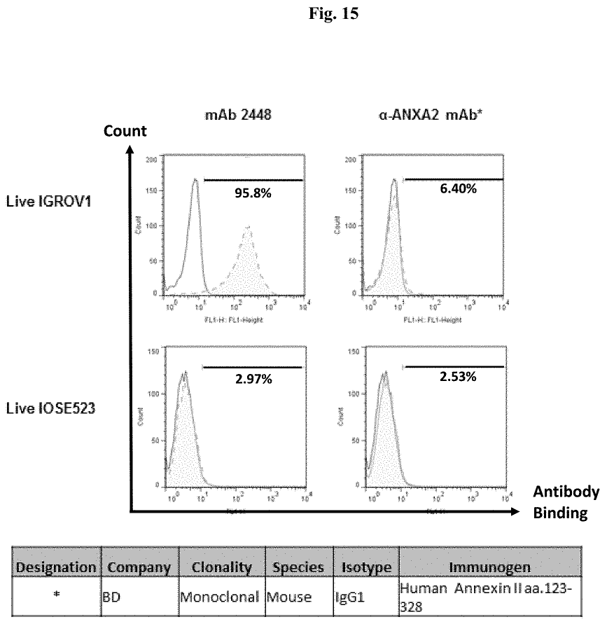

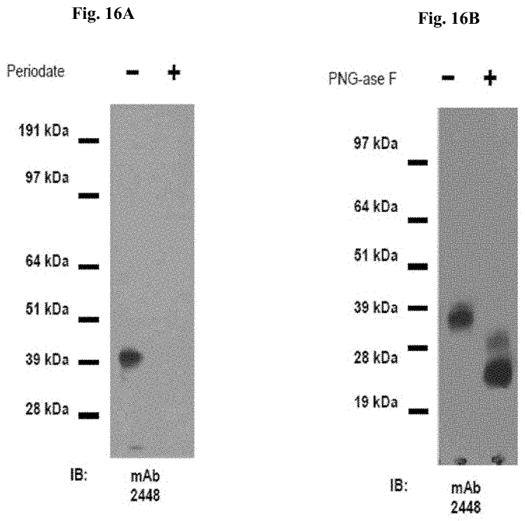

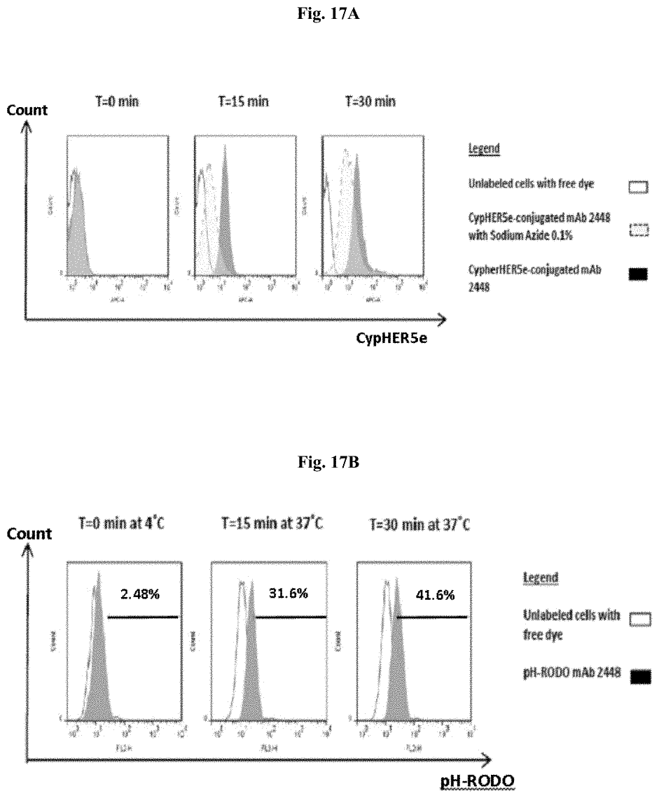

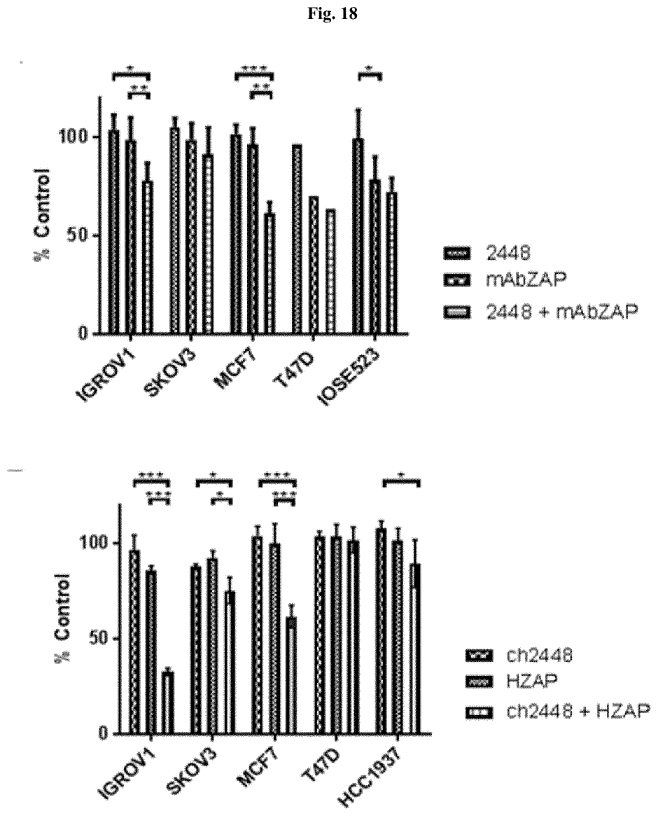

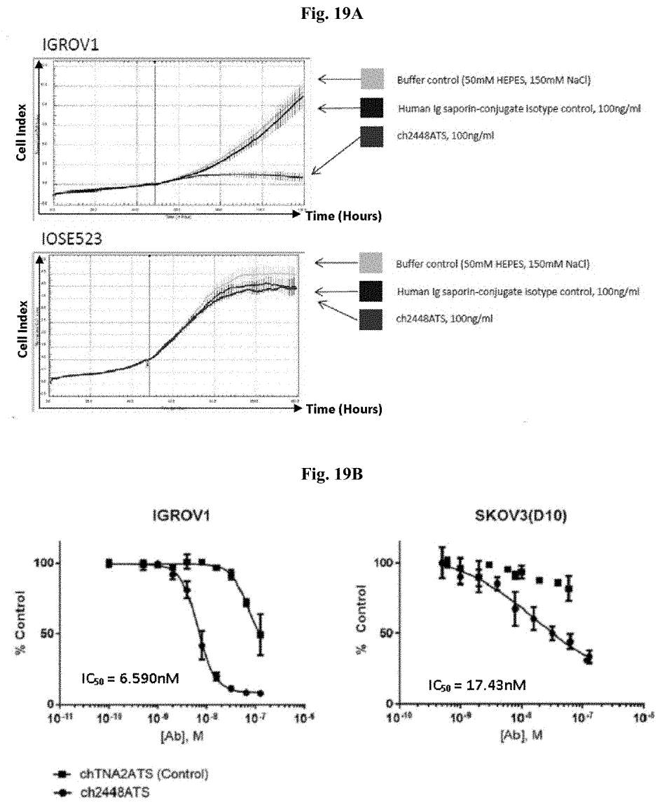

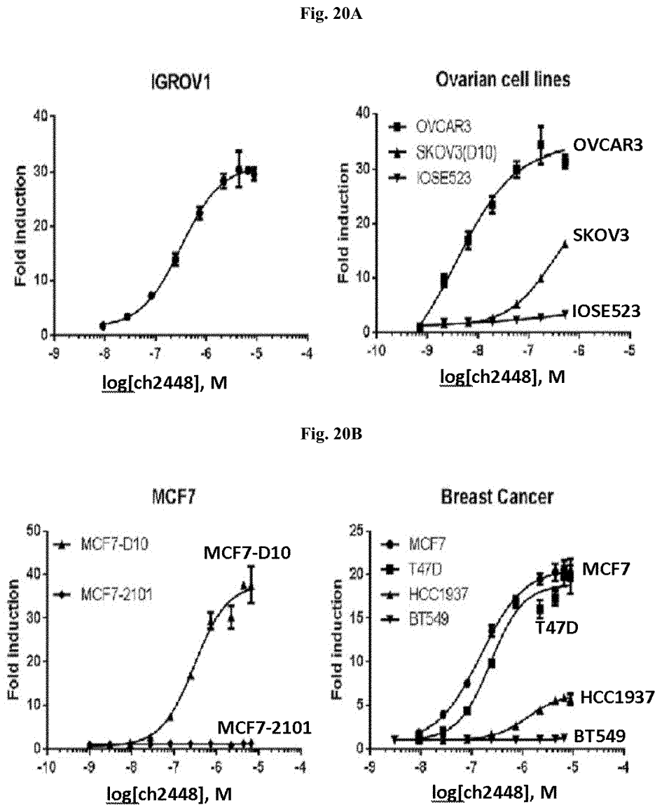

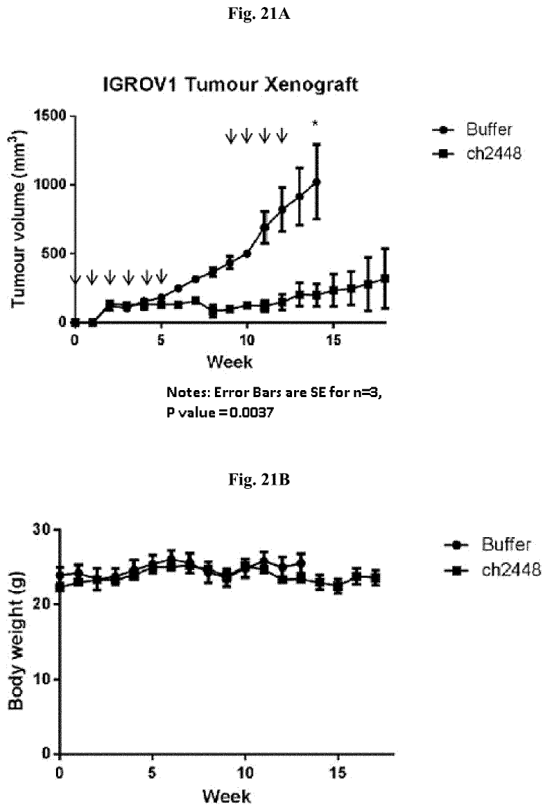

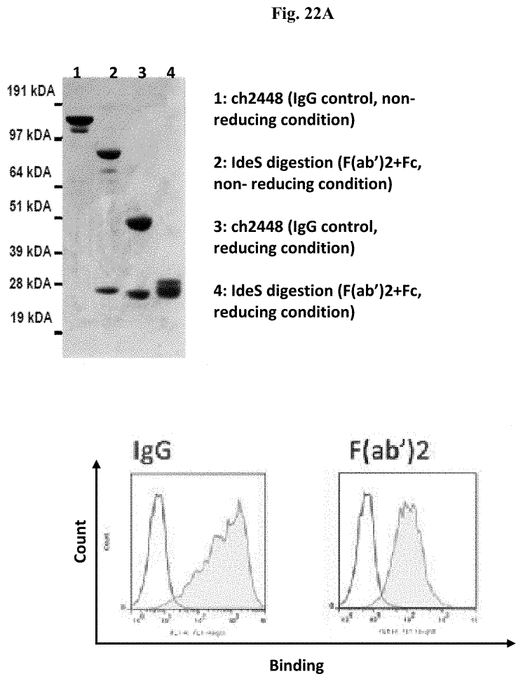

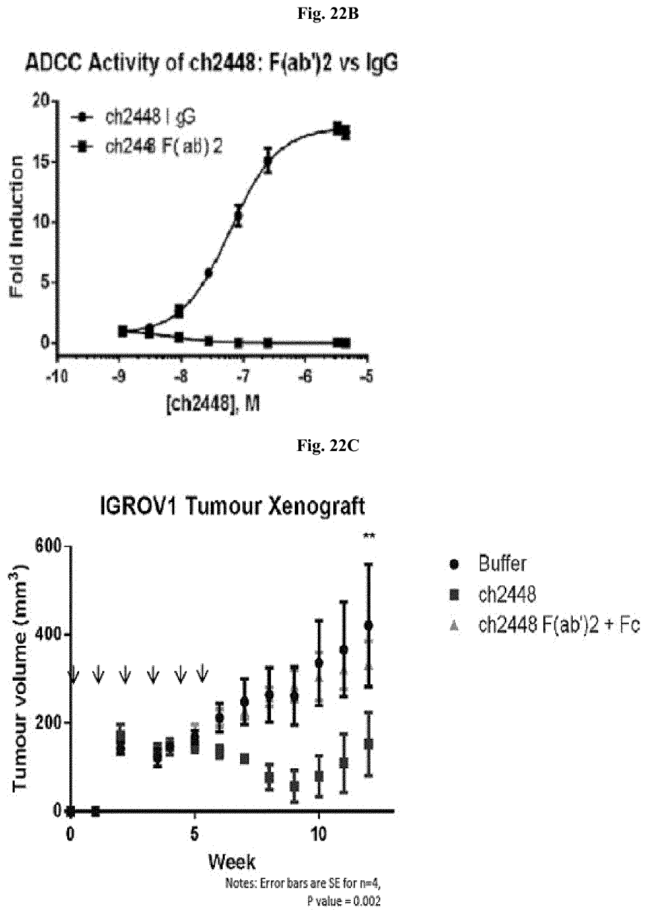

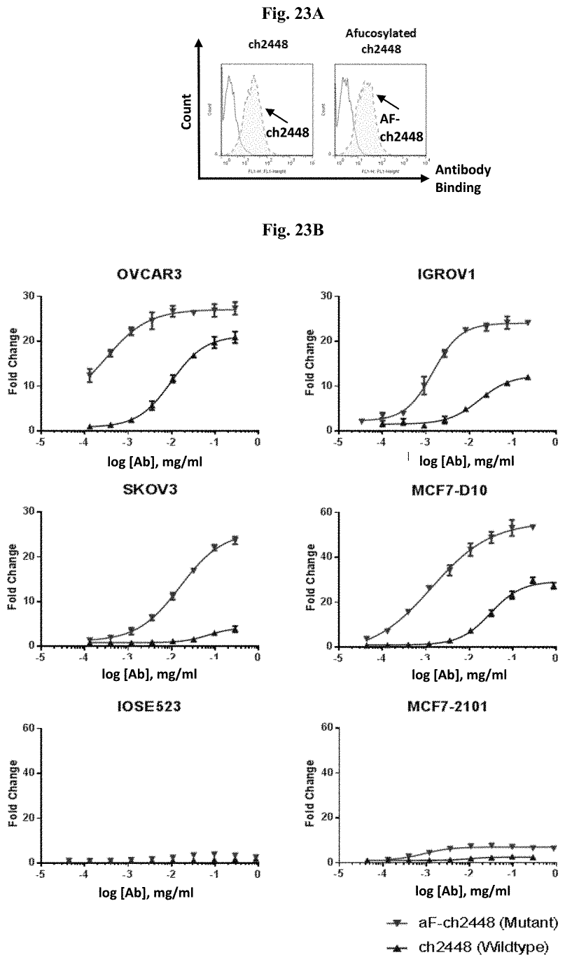

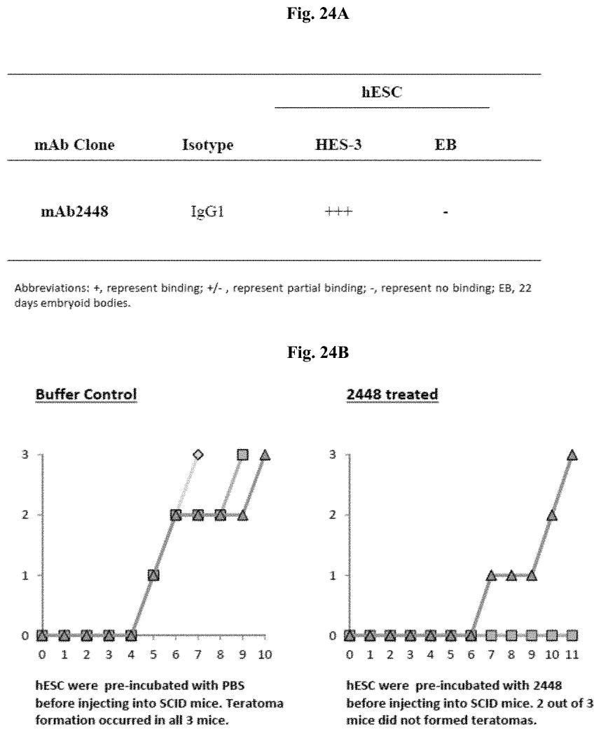

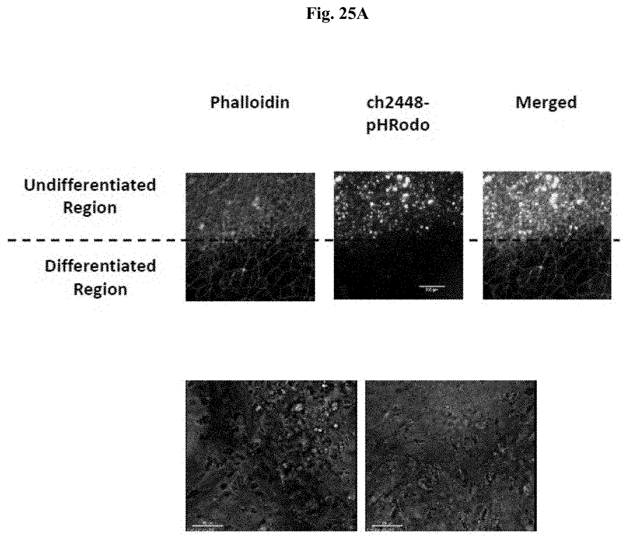

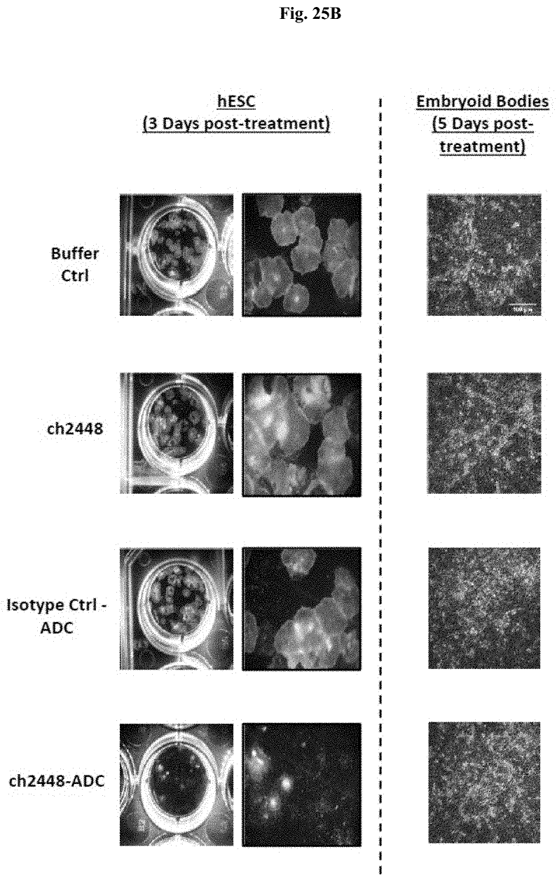

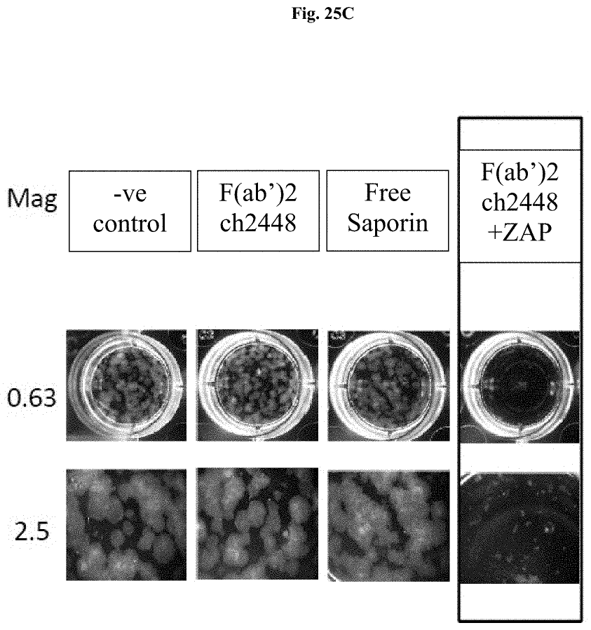

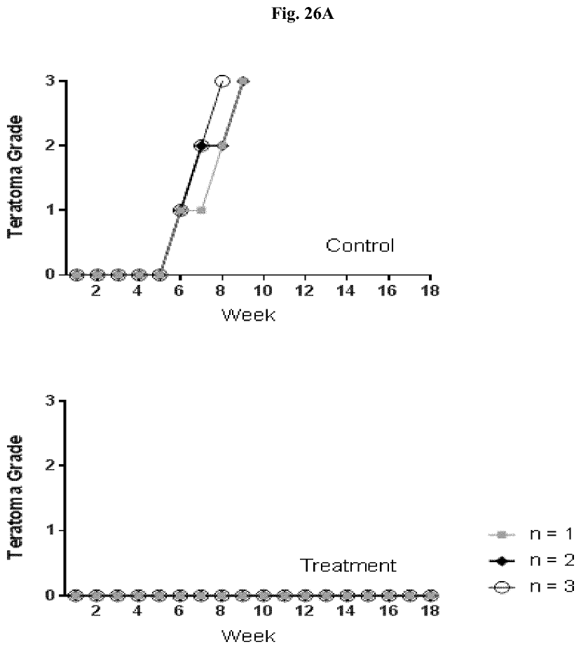

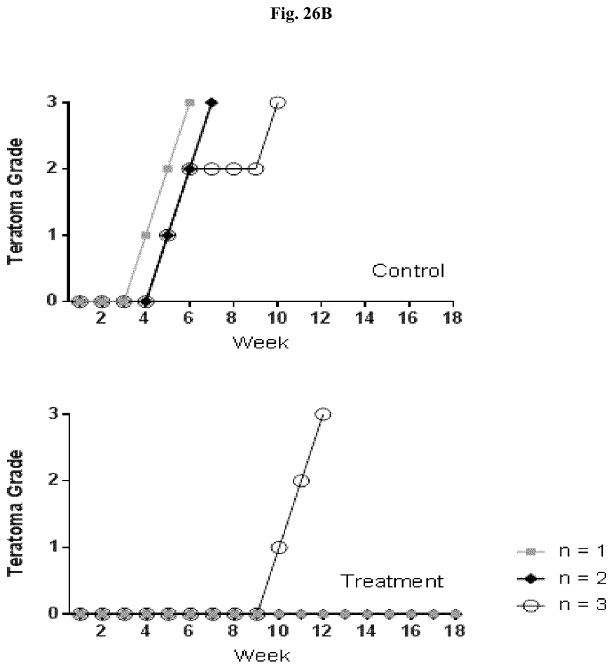

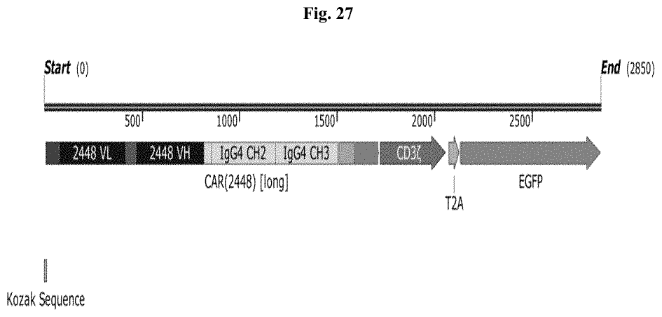

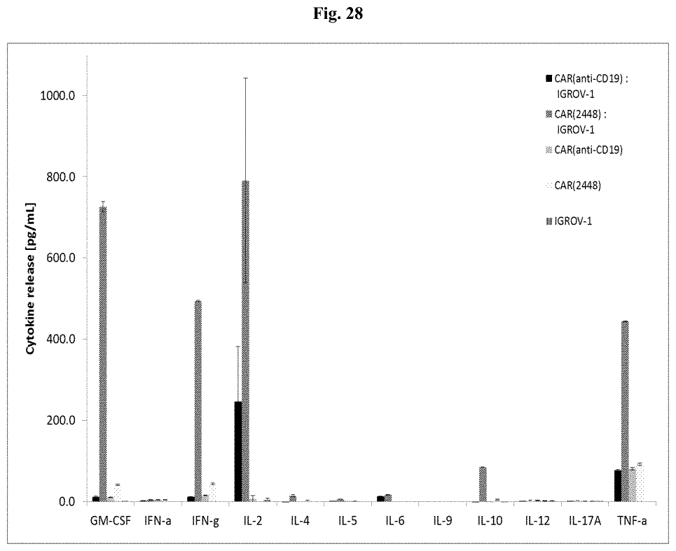

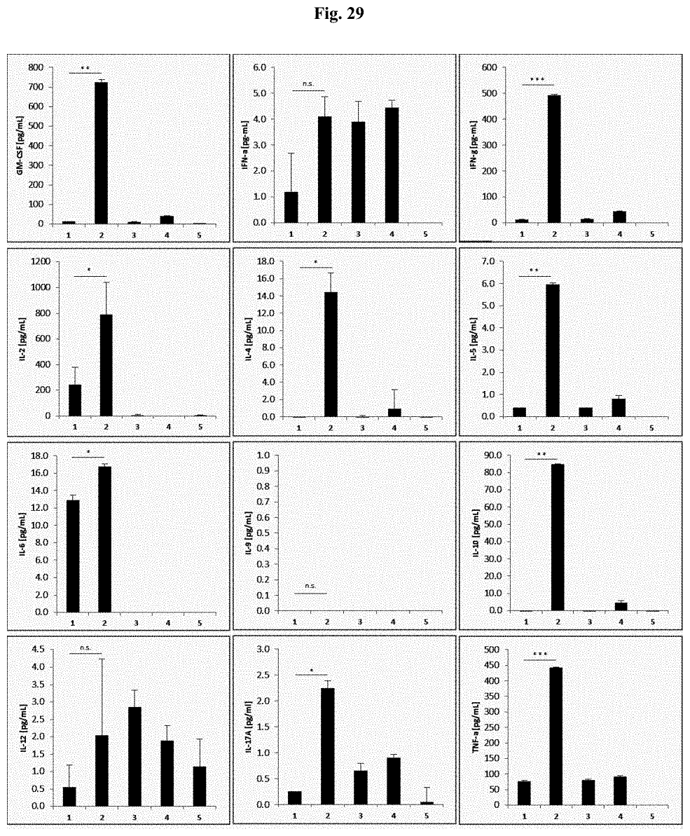

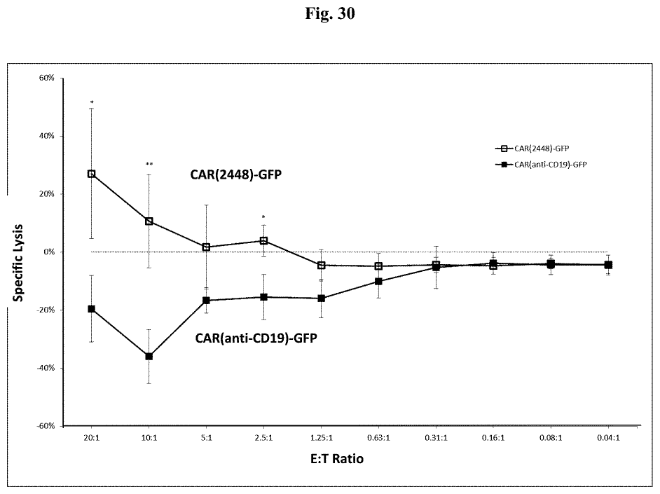

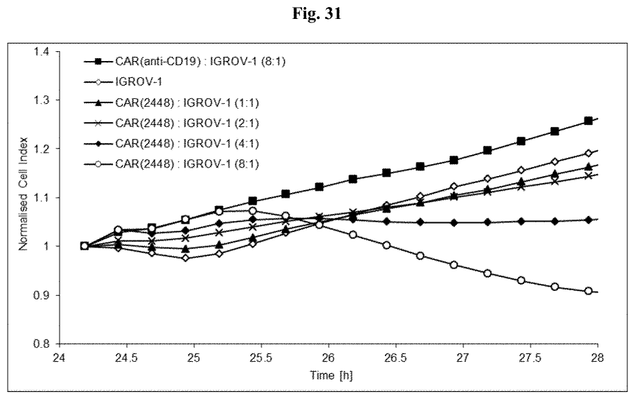

With this verification of the specificity of indel formation, RNPs and a single-stranded homology template (ssODN) were co-transfected into the same fibroblasts. The HT was further engineered to delete the PAM to prevent re-cutting and introduce a silent RFLP that would allow for efficient identification of HDR events (FIG. 11). HDR was confirmed at the population level by the presence of the RFLP in experimental samples (FIG. 14). Additionally, PCR designed using a 3′ primer specific for the HDR event (and a 5′ primer outside of the ssODN) amplified the target product in co-transfected cells, further indicating HDR had occurred (FIG. 15). While R408W was the primary sequence present in untreated fibroblasts, RNPs caused 50% reduction of the presence of the R408W sequence in amplicons sequenced from cells treated with or without ssODN (FIG. 16). Deep sequencing of the resulting amplicon showed disruption at 43% of then products present, with 5% being HDR and 38% 247 NHEJ (FIG. 17). Together, these data indicate the efficacy of these RNPs to initiate HDR to correct R408W in humanized sequences in these fibroblasts.

### DISCUSSION

A large animal model of phenylketonuria was developed with multiple research and drug/gene therapy development applications. This model shows several key similarities with the human disease that make it useful for basic and therapeutic research purposes, such as hyperphenylalaninemia, altered brain amino acid composition, hypopigmentation, and the ability to acutely control circulating Phe levels with dietary Phe restriction. As a general pig model of PKU, this animal is useful for the evaluation of disease progression and behavioral/dietary maintenance of subclinical Phe levels, as well as a useful model for the development of therapeutics for human consideration, including small molecules, enzyme replacement therapies, microbiome manipulations, etc.

Due to the humanization around the R408W mutation, this animal is a highly translatable model for gene editing for human PKU patients with at least one R408W allele, a prominent and debilitating isoform. Genetic targeting, such as via CRISPR or TALENs, intended for human use can be directly tested on this model without the need for development of disease model surrogates, which have varying relevance to the human product and are inefficient in both added cost and time.

Another interesting aspect to this model is the expression of PAH similar to wild type protein levels. Not only is this useful to model for any unanticipated effects of this expression in human disease and therapy, but it also reduces the potential for immunogenicity against a Pah transgene or the protein product of an edited Pah locus. In the case of R408W, function is eliminated by the substitution of a single amino acid in the transcript, which represents less than 0.3% variation from the expressed mutant protein. This theoretical attenuation of immunogenicity would be anticipated to benefit enzyme replacement therapy as well as both gene therapy approaches of gene delivery, which would add expression of a similar-yet-functional transgene, and gene editing, which would replace expression of some of the previously mutant translation product with functional PAH.

These R408W piglets are fragile in the neonatal period, and the fragility is difficult to completely ascribe to the PKU phenotype. Attempts were made in the earlier pregnancies to immediately attenuate circulating phenylalanine levels; however, as more pregnancies were supported the focus was placed entirely on maintaining piglet health since hyperphenylalaninemia is not acutely toxic in human patients, and this pig has characterized to be a high fidelity model of the human disease. This revised approach provided for the acute viability of a single founder. This animal has provided invaluable research materials such as primary PKU cells and data regarding maintenance of targeted circulating phenylalanine levels. Additional pregnancies are being performed to provide sufficient animals for herd breeding and preclinical testing of gene therapy and gene editing approaches.

Although the utility in PKU is appealing, the broader application of this animal model is the ability to show proof-of-concept for human gene editing platforms. This model can be investigated to demonstrate safety and efficacy of vectors and vehicles intended for human use to deliver human-translatable gene editing machinery in any gene editing platform.

The various examples and teachings described above are provided byway of illustration only and should not be construed to limit the scope of the present disclosure. Those skilled in the art will readily recognize various modifications and changes that may be made without following the examples and applications illustrated and described herein, and without departing from the true spirit and scope of the present disclosure. All publications referred to herein are incorporated by reference.

What is claimed is:

1. A method of introducing a mutation into a gene of interest in a non-human animal cell, the method comprising:
designing a targeted nuclease system to introduce double-stranded breaks in the DNA flanking the gene of interest;
designing a homology-directed repair (HDR) template oligonucleotide comprising a DNA sequence encoding a mutation in the gene of interest flanked by DNA sequences homologous to human DNA sequences flanking the mutation; and
delivering the targeted nuclease system and HDR template oligonucleotide into the non-human animal cell.

2. The method of claim 1, wherein the mutation causes loss of function of an enzyme.

3. The method of claim 1, wherein the mutation is a missense mutation.

4. The method of claim 1, wherein the missense mutation is commonly associated with a metabolic disorder caused by reduced or absent enzyme activity.

5. The method of claim 1, wherein the non-human animal is a mammal selected from the group comprising a swine, a bovine, a sheep, a goat, a horse, a deer, a primate, and a dog.

6. The method of claim 1, wherein the targeted nuclease system is selected from the group consisting of zinc finger nucleases, CRISPR/Cas9 endonucleases, and TAL effector nucleases.

7. The method of claim 1, wherein the double-stranded breaks occur no more than 50 nucleotides away from the gene of interest.

8. The method of claim 1, wherein at least one single nucleotide polymorphism (SNP) modifies the DNA at least 15 nucleotides upstream and downstream of the mutation to match human sequences.

9. The method of claim 1, wherein the cell is a somatic cell.

10. The method of claim 1, wherein the targeted nuclease system and HDR template oligonucleotide are delivered into the cell using at least one recombinant vector.

11. The method of claim 1, further comprising transferring DNA from the cell into an embryo of the non-human animal.

12. The method of claim 1, wherein the gene of interest is Pah.

13. The method of claim 12, wherein the HDR template oligonucleotide comprises a DNA sequence encoding a R408W mutation in exon 8.

14. The method of claim 1, wherein the DNA sequence comprises SEQ ID 16.

15. The method of claim 1, wherein the flanking regions are at least 20 nucleotides in length.

16. A recombinant vector comprising:
a polynucleotide encoding:
a targeted nuclease system designed to introduce double-stranded breaks in the DNA flanking a gene of interest in a non-human animal; and
a homology-directed repair (HDR) template having a DNA sequence including a mutation in the gene of interest that modifies its function and flanking sequences mutated to be homologous to human DNA flanking the location of the mutation.

17. The vector of claim 16, wherein the mutation causes a reduction or loss of function of the gene of interest.

18. The vector of claim 16, wherein the gene of interest is a gene associated with an inborn error of metabolism.

19. The vector of claim 18, wherein the inborn error of metabolism is phenylketonuria.

20. The vector of claim 16, wherein the flanking sequences are at least 15 nucleotides long.

21. The vector of claim 16, wherein the recombinant vector is a viral vector selected from the group comprising: retroviral vector, lentiviral vector, adenoviral vector, and adeno-associated vector.

22. The vector of claim 16, wherein the non-human animal is an ungulate.

23. A non-human mammal model of an inborn error of metabolism, the model having a DNA sequence comprising:
a mutation in a gene that causes the inborn error of metabolism, and
two or more single nucleotide polymorphisms (SNPs) in the DNA flanking the mutation that causes the DNA to match a homologous human DNA sequence.

24. The non-human mammal model of claim 23, wherein the inborn error of metabolism is one of adrenoleukodystrophy, Gaucher disease, hereditary hemochromatosis, Lesch-Nyhan syndrome, Maple syrup urine disease, Glucose galactose malabsorption disease, Menkes syndrome, Niemann-Pick disease, phenylketonuria, Refsum disease, Tangier disease, Tay-Sachs disease, Wilson's disease, Zellweger syndrome, Alkaptonuria, Carnosinemia, Cystinuria, fumaric aciduria, Tyrosinemia, Sarcosinemia, Hyperlysinemia, and Hyperprolinemia.

25. The non-human mammal model of claim 23, wherein the non-human mammal is a pig and the inborn error of metabolism is phenylketonuria (PKU).
